(19) United States
(12) Patent Application Publication
(10) Pub. No.: US 2023/0272435 A1
(43) Pub. Date: Aug. 31, 2023

(54) DISCOVERY AND ENGINEERING OF INTEGRASES FOR HIGH-EFFICIENCY GENE INTEGRATION

(71) Applicant: Massachusetts Institute of Technology, Cambridge, MA (US)

(72) Inventors: Omar Abudayyeh, Cambridge, MA (US); Jonathan Gootenberg, Cambridge, MA (US); Lukas Villiger, Cambridge, MA (US); Justin Lim, Boston, MA (US)

(21) Appl. No.: 18/048,238

(22) Filed: Oct. 20, 2022

Related U.S. Application Data
(60) Provisional application No. 63/262,829, filed on Oct. 21, 2021.
(60) Provisional application No. 63/265,002, filed on Dec. 6, 2021.

(51) Int. Cl. C12N 15/90; C12N 9/22; C12N 15/11; C12N 9/12

(52) U.S. Cl. CPC C12N 15/907; C12N 9/22; C12N 15/111; C12N 9/1276; C12N 2310/20; C12N 2800/30; C07K 2319/80

(57) ABSTRACT

This disclosure provides novel integrases for site-specific genetic engineering. Also provided are systems, methods, and compositions for site-specific genetic engineering using Programmable Addition via Site-Specific Targeting Elements (PASTE) with the novel integrases.

### RELATED APPLICATIONS

This application claims the benefit of U.S. Provisional Patent Application Ser. Nos. 63/262,829 and 63/265,002 respectively filed on Oct. 21, 2021, and Dec. 6, 2021, the entire disclosures of which are incorporated herein by reference.

### SEQUENCE LISTING

The instant application contains a Sequence Listing which has been submitted electronically in XML file format and is hereby incorporated by reference in its entirety. Said XML copy, created on Oct. 20, 2022, is named 733335_083474-027 SL.xml and is 73,894,000 bytes in size.

### STATEMENT REGARDING FEDERALLY SPONSORED RESEARCH OR DEVELOPMENT

This invention was made with Government support under Grant No. R21 A1149694 awarded by the National Institutes of Health. The Government has certain rights in the invention.

### BACKGROUND

Integrases provide efficient modes for non-programmable genome integration. Site-specific integrases, such as large serine phage integrases, efficiently integrate their DNA cargo into sequence-defined landing sites that are about 30-50 nucleotides long and can be used to insert therapeutic transgenes at naturally occurring pseudo-sites in the human genome in pre-clinical models (Brown et al. Methods. 2011. 53: 372-379; Calos. Curr. Gene Ther. 2006. 6: 633-645). Targeted integration can also be achieved by a two-step approach involving prior insertion of integrase landing sites at a desired location using homology-directed repair (HDR) (Mulholland et al. Nucleic Acids Res. 2015. 43: e112). However, the inefficiency of two-step integration with HDR and the risks associated with double-strand breaks (DSBs) have limited this approach in mammalian cells. Furthermore, a major issue limiting clinical application of certain integrases, such as phiC31, is that chromosomal rearrangements between pseudo-sites can occur, leading to a significant DNA damage response (Ehrhardt et al. Hum. Gene Ther. 2006. 17: 1077-1094; Liu et al. Gene Ther. 2006. 13: 1188-1190).

Accordingly, there exists a need for novel integrases that are compatible as fusions with programmable DNA binding proteins.

### SUMMARY

In one aspect, the disclosure provides an integrase or fragment thereof comprising an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in any one of SEQ ID NOs: 1-16.

In certain embodiments, the integrase fragment comprises integrase, recombinase, or transposase activity.

In certain embodiments, the integrase or fragment thereof comprises one or more mutations.

In certain embodiments, the integrase or fragment thereof is encoded by a codon optimized nucleic acid sequence. In certain embodiments, the nucleic acid sequence is codon optimized for expression in humans.

In certain embodiments, the integrase binds to nucleic acid attachment sites attB and attP, other recognition site pairs, or any pseudosites in a human genome.

In certain embodiments, the attB and/or attP nucleic acid sequence is between 12 and 60 nucleotides in length or between 18 and 50 nucleotides in length.

In certain embodiments, the attB and/or attP nucleic acid sequence comprises one or more truncations.

In certain embodiments, the attB and/or attP nucleic acid sequence is truncated by 1 to 32 nucleotides from one or both of the 5′ end and 3′ end.

In certain embodiments, the integrase binds to any one of the attB nucleic acid sequences selected from the group consisting of SEQ ID NOs: 17, 19, 21, 23, 25, 27, 29, 31, 33, 35, 37, 39, 41, 43, 45, and 47.

In certain embodiments, the integrase binds to any one of the attP nucleic acid sequences selected from the group consisting of SEQ ID NOs: 18, 20, 22, 24, 26, 28, 30, 32, 34, 36, 38, 40, 42, 44, 46, and 48.

In certain embodiments: a) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 1, wherein the integrase binds to the attB nucleic acid set forth in SEQ ID NO: 17 and the attP nucleic acid set forth in SEQ ID NO: 18; b) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 2, wherein the integrase binds to the attB nucleic acid set forth in SEQ ID NO: 19 and the attP nucleic acid set forth in SEQ ID NO: 20; c) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 3, wherein the integrase binds to the attB nucleic acid set forth in SEQ ID NO: 21 and the attP nucleic acid set forth in SEQ ID NO: 22; d) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 4, wherein the integrase binds to the attB nucleic acid set forth in SEQ ID NO: 23 and the attP nucleic acid set forth in SEQ ID NO: 24; e) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 5, wherein the integrase binds to the attB nucleic acid set forth in SEQ ID NO: 25 and the attP nucleic acid set forth in SEQ ID NO: 26; f) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 6, wherein the integrase binds to the attB nucleic acid set forth in SEQ ID NO: 27 and the attP nucleic acid set forth in SEQ ID NO: 28; g) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 7, wherein the integrase binds to the attB nucleic acid set forth in SEQ ID NO: 29 and the attP nucleic acid set forth in SEQ ID NO: 30; h) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 8, wherein the integrase binds to the attB nucleic acid set forth in SEQ ID NO: 31 and the attP nucleic acid set forth in SEQ ID NO: 32; i) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 9, wherein the integrase binds to the attB nucleic acid set forth in SEQ ID NO: 33 and the attP nucleic acid set forth in SEQ ID NO: 34; j) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 10, wherein the integrase binds to the attB nucleic acid set forth in SEQ ID NO: 35 and the attP nucleic acid set forth in SEQ ID NO: 36; k) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 11, wherein the integrase binds to the attB nucleic acid set forth in SEQ ID NO: 37 and the attP nucleic acid set forth in SEQ ID NO: 38; 1) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 12, wherein the integrase binds to the attB nucleic acid set forth in SEQ ID NO: 39 and the attP nucleic acid set forth in SEQ ID NO: 40; m) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 13, wherein the integrase binds to the attB nucleic acid set forth in SEQ ID NO: 41 and the attP nucleic acid set forth in SEQ ID NO: 42; n) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 14, wherein the integrase binds to the attB nucleic acid set forth in SEQ ID NO: 43 and the attP nucleic acid set forth in SEQ ID NO: 44; o) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 15, wherein the integrase binds to the attB nucleic acid set forth in SEQ ID NO: 45 and the attP nucleic acid set forth in SEQ ID NO: 46; or p) the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 16, wherein the integrase binds to the attB nucleic acid set forth in SEQ ID NO: 47 and the attP nucleic acid set forth in SEQ ID NO: 48.

In certain embodiment, any one of the attB nucleic acid sequences selected from the group consisting of SEQ ID NOs: 17, 19, 21, 23, 25, 27, 29, 31, 33, 35, 37, 39, 41, 43, 45, and 47 is truncated by 1 to 32 nucleotides (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 nucleotides) from one or both of the 5′ end and 3′ end.

In certain embodiment, any one of the attP nucleic acid sequences selected from the group consisting of SEQ ID NOs: 18, 20, 22, 24, 26, 28, 30, 32, 34, 36, 38, 40, 42, 44, 46, and 48 is truncated by 1 to 32 nucleotides (i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 nucleotides) from one or both of the 5′ end and 3′ end.

In certain embodiment, the integrase is linked to a DNA binding domain via a linker. In certain embodiment, the DNA binding domain is a DNA binding nuclease.

In certain embodiment, the DNA binding nuclease is selected from the group consisting of a zinc finger nuclease (ZFN), a transcription-activator like effector nuclease (TALEN), an argonaute, and an RNA-guided nuclease.

In certain embodiment, the RNA-guided nuclease comprises a CRISPR nuclease.

In certain embodiment, the CRISPR nuclease is Cas9 or Cas12.

In certain embodiment, the CRISPR nuclease comprises nickase activity.

In certain embodiment, the CRISPR nuclease is selected from Cas9-D10A, Cas9-H840A, and Cas12a/b nickase.

In certain embodiment, the DNA binding nuclease and/or the integrase are linked to a reverse transcriptase domain, via a linker.

In certain embodiment, the reverse transcriptase domain is selected from the group consisting of Moloney Murine Leukemia Virus (M-MLV) reverse transcriptase domain, transcription xenopolymerase (RTX), avian myeloblastosis virus reverse transcriptase (AMV-RT), and Eubacterium rectale maturase RT (MarathonRT).

In certain embodiment, the reverse transcriptase domain comprises a mutation relative to the wild-type sequence or contains a stabilization domain like the DNA-binding Sto7d protein from Sulfolobus tokodaii.

In certain embodiment, the M-MLV reverse transcriptase domain comprises one or more mutations selected from the group consisting of D200N, T306K, W313F, T330P, L603W, and L139P.

In certain embodiment, the linker is cleavable.

In certain embodiment, the linker is non-cleavable.

In certain embodiment, the linker can be replaced by two associating binding domains of the DNA binding nuclease linked to a reverse transcriptase.

In certain embodiment, the DNA binding nuclease interacts with a guide RNA (gRNA) comprising a primer binding sequence linked to an integration sequence.

In certain embodiment, the gRNA interacts with the DNA binding nuclease and targets a desired location in a cell genome.

In certain embodiment, the DNA binding nuclease nicks a strand of the cell genome and the reverse transcriptase domain incorporates the integration sequence of the gRNA into the nicked site, thereby providing the integration site at the desired location of the cell genome.

In certain embodiment, the integrase is capable of binding the integration sequence.

In one aspect, the disclosure provides a polynucleotide comprising a nucleic acid sequence encoding the integrase described above.

In one aspect, the disclosure provides a vector comprising the nucleic acid sequence described above.

In one aspect, the disclosure provides a host cell comprising the vector described above.

In one aspect, the disclosure provides a fusion protein comprising DNA binding nuclease, a reverse transcriptase domain, and an integrase or fragment thereof, wherein the DNA binding nuclease is linked to the reverse transcriptase domain and/or the integrase or fragment thereof via a linker, wherein the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in any one of SEQ ID NOs: 1-16.

In one aspect, the disclosure provides a fusion protein comprising DNA binding nuclease, a reverse transcriptase domain, and an integrase or fragment thereof, wherein the DNA binding nuclease is linked to the reverse transcriptase domain and/or the integrase or fragment thereof via a linker, wherein the integrase or fragment thereof comprises an amino acid sequence that is at least 80% (i.e., 80%, 81%, 82%, 83%, 84%, 85%, 86%, 87%, 88%, 89%, 90%, 91%, 92%, 93%, 94%, 95%, 96%, 97%, 99%, or 100%) identical to an amino acid sequence set forth in SEQ ID NO: 14.

In certain embodiments of the fusion protein, the integrase fragment comprises integrase activity.

In certain embodiments of the fusion protein, the fusion protein is encoded by a codon optimized nucleic acid sequence. In certain embodiments of the fusion protein, the nucleic acid sequence is codon optimized for expression in humans.

In certain embodiments of the fusion protein, the integrase further comprises one or more mutations.

In certain embodiments of the fusion protein, the integrase binds to nucleic acid attachment sites attB and attP, other recognition site pairs, or any pseudosites in the human genome.

In certain embodiments of the fusion protein, the attB and/or attP nucleic acid sequence is between 12 and 60 nucleotides in length or between 18 and 50 nucleotides in length.

In certain embodiments of the fusion protein, the attB and/or attP nucleic acid sequence comprises one or more truncations.

In certain embodiments of the fusion protein, the attB and/or attP nucleic acid sequence is truncated by 1 to 32 nucleotides from one or both of the 5′ end and 3′ end.

In certain embodiments of the fusion protein, the integrase binds to any one of the attB nucleic acid sequences selected from the group consisting of SEQ ID NOs: 17, 19, 21, 23, 25, 27, 29, 31, 33, 35, 37, 39, 41, 43, 45, and 47.

In certain embodiments of the fusion protein, the integrase binds to any one of the attP nucleic acid sequences selected from the group consisting of SEQ ID NOs: 18, 20, 22, 24, 26, 28, 30, 32, 34, 36, 38, 40, 42, 44, 46, and 48.

In certain embodiments of the fusion protein: a) the integrase or fragment thereof comprises an amino acid sequence that is at least 80% identical to an amino acid sequence set forth in SEQ ID NO: 1, wherein the integrase binds to the attB nucleic acid set forth in SEQ ID NO: 17 and the attP nucleic acid set forth in SEQ ID NO: 18; b) the integrase or fragment thereof comprises an amino acid sequence that is at least 80% identical to an amino acid sequence set forth in SEQ ID NO: 2, wherein the integrase binds to the attB nucleic acid set forth in SEQ ID NO: 19 and the attP nucleic acid set forth in SEQ ID NO: 20; c) the integrase or fragment thereof comprises an amino acid sequence that is at least 80% identical to an amino acid sequence set forth in SEQ ID NO: 3, wherein the integrase binds to the attB nucleic acid set forth in SEQ ID NO: 21 and the attP nucleic acid set forth in SEQ ID NO: 22; d) the integrase or fragment thereof comprises an amino acid sequence that is at least 80% identical to an amino acid sequence set forth in SEQ ID NO: 4, wherein the integrase binds to the attB nucleic acid set forth in SEQ ID NO: 23 and the attP nucleic acid set forth in SEQ ID NO: 24; e) the integrase or fragment thereof comprises an amino acid sequence that is at least 80% identical to an amino acid sequence set forth in SEQ ID NO: 5, wherein the integrase binds to the attB nucleic acid set forth in SEQ ID NO: 25 and the attP nucleic acid set forth in SEQ ID NO: 26; f) the integrase or fragment thereof comprises an amino acid sequence that is at least 80% identical to an amino acid sequence set forth in SEQ ID NO: 6, wherein the integrase binds to the attB nucleic acid set forth in SEQ ID NO: 27 and the attP nucleic acid set forth in SEQ ID NO: 28; g) the integrase or fragment thereof comprises an amino acid sequence that is at least 80% identical to an amino acid sequence set forth in SEQ ID NO: 7, wherein the integrase binds to the attB nucleic acid set forth in SEQ ID NO: 29 and the attP nucleic acid set forth in SEQ ID NO: 30; h) the integrase or fragment thereof comprises an amino acid sequence that is at least 80% identical to an amino acid sequence set forth in SEQ ID NO: 8, wherein the integrase binds to the attB nucleic acid set forth in SEQ ID NO: 31 and the attP nucleic acid set forth in SEQ ID NO: 32; i) the integrase or fragment thereof comprises an amino acid sequence that is at least 80% identical to an amino acid sequence set forth in SEQ ID NO: 9, wherein the integrase binds to the attB nucleic acid set forth in SEQ ID NO: 33 and the attP nucleic acid set forth in SEQ ID NO: 34; j) the integrase or fragment thereof comprises an amino acid sequence that is at least 80% identical to an amino acid sequence set forth in SEQ ID NO: 10, wherein the integrase binds to the attB nucleic acid set forth in SEQ ID NO: 35 and the attP nucleic acid set forth in SEQ ID NO: 36; k) the integrase or fragment thereof comprises an amino acid sequence that is at least 80% identical to an amino acid sequence set forth in SEQ ID NO: 11, wherein the integrase binds to the attB nucleic acid set forth in SEQ ID NO: 37 and the attP nucleic acid set forth in SEQ ID NO: 38; 1) the integrase or fragment thereof comprises an amino acid sequence that is at least 80% identical to an amino acid sequence set forth in SEQ ID NO: 12, wherein the integrase binds to the attB nucleic acid set forth in SEQ ID NO: 39 and the attP nucleic acid set forth in SEQ ID NO: 40; m) the integrase or fragment thereof comprises an amino acid sequence that is at least 80% identical to an amino acid sequence set forth in SEQ ID NO: 13, wherein the integrase binds to the attB nucleic acid set forth in SEQ ID NO: 41 and the attP nucleic acid set forth in SEQ ID NO: 42; n) the integrase or fragment thereof comprises an amino acid sequence that is at least 80% identical to an amino acid sequence set forth in SEQ ID NO: 14, wherein the integrase binds to the attB nucleic acid set forth in SEQ ID NO: 43 and the attP nucleic acid set forth in SEQ ID NO: 44; o) the integrase or fragment thereof comprises an amino acid sequence that is at least 80% identical to an amino acid sequence set forth in SEQ ID NO: 15, wherein the integrase binds to the attB nucleic acid set forth in SEQ ID NO: 45 and the attP nucleic acid set forth in SEQ ID NO: 46; or p) the integrase or fragment thereof comprises an amino acid sequence that is at least 80% identical to an amino acid sequence set forth in SEQ ID NO: 16, wherein the integrase binds to the attB nucleic acid set forth in SEQ ID NO: 47 and the attP nucleic acid set forth in SEQ ID NO: 48.

In certain embodiments of the fusion protein, the integrase or fragment thereof comprises an amino acid sequence that is at least 80% identical to an amino acid sequence set forth in SEQ ID NO: 14, wherein the integrase binds to the attB nucleic acid set forth in SEQ ID NO: 43 and the attP nucleic acid set forth in SEQ ID NO: 44.

In certain embodiments of the fusion protein, any one of the attB nucleic acid sequences selected from the group consisting of SEQ ID NOs: 17, 19, 21, 23, 25, 27, 29, 31, 33, 35, 37, 39, 41, 43, 45, and 47 is truncated by 1 to 32 nucleotides from one or both of the 5′ end and 3′ end.

In certain embodiments of the fusion protein, any one of the attP nucleic acid sequences selected from the group consisting of SEQ ID NOs: 18, 20, 22, 24, 26, 28, 30, 32, 34, 36, 38, 40, 42, 44, 46, and 48 is truncated by 1 to 32 nucleotides from one or both of the 5′ end and 3′ end.

In certain embodiments of the fusion protein, the integrase is linked to a DNA binding domain via a linker.

In certain embodiments of the fusion protein, the DNA binding domain is a DNA binding nuclease.

In certain embodiments of the fusion protein, the DNA binding nuclease is selected from the group consisting of a zinc finger nuclease (ZFN), a transcription-activator like effector nuclease (TALEN), and an RNA-guided nuclease.

In certain embodiments of the fusion protein, the RNA-guided nuclease comprises a CRISPR nuclease.

In certain embodiments of the fusion protein, the CRISPR nuclease is Cas9 or Cas12.

In certain embodiments of the fusion protein, the CRISPR nuclease comprises nickase activity.

In certain embodiments of the fusion protein, the CRISPR nuclease is selected from Cas9-D10A, Cas9-H840A, and Cas12a/b nickase.

In certain embodiments of the fusion protein, the reverse transcriptase domain is selected from the group consisting of Moloney Murine Leukemia Virus (M-MLV) reverse transcriptase domain, transcription xenopolymerase (RTX), avian myeloblastosis virus reverse transcriptase (AMV-RT), and Eubacterium rectale maturase RT (MarathonRT).

In certain embodiments of the fusion protein, the reverse transcriptase domain comprises a mutation relative to the wild-type sequence or contains a stabilization domain like the DNA-binding Sto7d protein from Sulfolobus tokodaii.

In certain embodiments of the fusion protein, the M-MLV reverse transcriptase domain comprises one or more mutations selected from the group consisting of D200N, T306K, W313F, T330P, L603W, and L139P.

In certain embodiments of the fusion protein, the linker is cleavable. In certain embodiments of the fusion protein, the linker is non-cleavable.

In certain embodiments of the fusion protein, the linker can be replaced by two associating binding domains of the DNA binding nuclease linked to a reverse transcriptase.

In certain embodiments of the fusion protein, the DNA binding nuclease interacts with a guide RNA (gRNA) comprising a primer binding sequence linked to an integration sequence.

In certain embodiments of the fusion protein, the gRNA interacts with the DNA binding nuclease and targets a desired location in a cell genome.

In certain embodiments of the fusion protein, the DNA binding nuclease nicks a strand of the cell genome and the reverse transcriptase domain incorporates the integration sequence of the gRNA into the nicked site, thereby providing the integration site at the desired location of the cell genome.

In certain embodiments of the fusion protein, the integrase is capable of binding the integration sequence.

In one aspect, the disclosure provides a polynucleotide comprising a nucleic acid sequence encoding the fusion protein described above.

In one aspect, the disclosure provides a vector comprising the nucleic acid sequence described above.

In one aspect, the disclosure provides a host cell comprising the vector described above.

In one aspect, the disclosure provides a method of site-specific integration of a nucleic acid into a cell genome, the method comprising:
(a) incorporating an integration site at a desired location in the cell genome by introducing into the cell:
   i. a DNA binding nuclease linked to a reverse transcriptase domain, wherein the DNA binding nuclease comprises a nickase activity; and
   ii. a guide RNA (gRNA) comprising a primer binding sequence linked to an integration sequence, wherein the gRNA interacts with the DNA binding nuclease and targets the desired location in the cell genome, wherein the DNA binding nuclease nicks a strand of the cell genome and the reverse transcriptase domain incorporates the integration sequence of the gRNA into the nicked site, thereby providing the integration site at the desired location of the cell genome; and
(b) integrating the nucleic acid into the cell genome by introducing into the cell:
   i. a DNA or RNA strand comprising the nucleic acid linked to a sequence that is complementary or associated to the integration site; and
   ii. an integrase or fragment thereof comprising an amino acid sequence that is at least 80% identical to an amino acid sequence set forth in any one of SEQ ID NOs: 1-16, wherein the integrase or fragment thereof incorporates the nucleic acid into the cell genome at the integration site by integration, recombination, or reverse transcription of the sequence that is complementary or associated to the integration site, thereby introducing the nucleic acid into the desired location of the cell genome of the cell.

In one aspect, the disclosure provides a method of site-specific integration of a nucleic acid into a cell genome, the method comprising:
(a) incorporating an integration site at a desired location in the cell genome by introducing into the cell:
   i. a DNA binding nuclease linked to a reverse transcriptase domain, wherein the DNA binding nuclease comprises a nickase activity; and
   ii. a guide RNA (gRNA) comprising a primer binding sequence linked to an integration sequence, wherein the gRNA interacts with the DNA binding nuclease and targets the desired location in the cell genome, wherein the DNA binding nuclease nicks a strand of the cell genome and the reverse transcriptase domain incorporates the integration sequence of the gRNA into the nicked site, thereby providing the integration site at the desired location of the cell genome; and
(b) integrating the nucleic acid into the cell genome by introducing into the cell:
   i. a DNA or RNA strand comprising the nucleic acid linked to a sequence that is complementary or associated to the integration site; and
   ii. an integrase or fragment thereof comprising an amino acid sequence that is at least 80% identical to an amino acid sequence set forth in 14, wherein the integrase or fragment thereof incorporates the nucleic acid into the cell genome at the integration site by integration, recombination, or reverse transcription of the sequence that is complementary or associated to the integration site, thereby introducing the nucleic acid into the desired location of the cell genome of the cell.

In certain embodiments, the gRNA hybridizes to a complementary strand of the cell genome to the genomic strand that is nicked by the DNA binding nuclease.

In certain embodiments, the integrase is introduced as a polypeptide or a nucleic acid encoding the same.

In certain embodiments, the DNA binding nuclease is introduced as a polypeptide or a nucleic acid encoding the same.

In certain embodiments, the DNA or RNA strand comprising the nucleic acid is introduced into the cell as a minicircle, a plasmid, mRNA or a linear DNA.

In certain embodiments, the DNA or RNA strand comprising the nucleic acid is between 1000 bp and 10,000 bp.

In certain embodiments, the DNA or RNA strand comprising the nucleic acid is more than 10,000 bp.

In certain embodiments, the DNA or RNA strand comprising the nucleic acid is less than 1000 bp.

In certain embodiments, the DNA or RNA strand comprising the nucleic acid is introduced into the cell with a viral vector.

In certain embodiments, the viral vector is an AAV vector, an adenoviral vector, or a lentiviral vector.

In certain embodiments, the DNA comprising the nucleic acid is introduced into the cell as a minicircle.

In certain embodiments, the minicircle does not comprise sequences of a bacterial origin.

In certain embodiments, the DNA binding nuclease linked to a reverse transcriptase domain and the integrase are linked via a linker.

In certain embodiments, the linker is cleavable. In certain embodiments, the linker is non-cleavable.

In certain embodiments, the linker can be replaced by two associating binding domains of the DNA binding nuclease linked to a reverse transcriptase.

In certain embodiments, the integration site is selected from an attB site, an attP site, an attL site, or an attR site.

In certain embodiments, the DNA binding nuclease comprising a nickase activity is selected from Cas9-D10A, Cas9-H840A, and Cas12a/b nickase.

In certain embodiments, the reverse transcriptase domain is selected from the group consisting of Moloney Murine Leukemia Virus (M-MLV) reverse transcriptase domain, transcription xenopolymerase (RTX), avian myeloblastosis virus reverse transcriptase (AMV-RT), and Eubacterium rectale maturase RT (MarathonRT).

In certain embodiments, the reverse transcriptase domain comprises a mutation relative to the wild-type sequence or contains a stabilization domain like the DNA-binding Sto7d protein from Sulfolobus tokodaii.

In certain embodiments, the M-MLV reverse transcriptase domain comprises one or more mutations selected from the group consisting of D200N, T306K, W313F, T330P L603W, and L139P.

In certain embodiments, the method of further comprises introducing a second nicking guide RNA (ngRNA).

In certain embodiments, the gRNA, the nucleic acid encoding the DNA binding nuclease, the reverse transcriptase, the DNA comprising nucleic acid linked to a complementary or associated integration site, the integrase, and optionally the ngRNA are introduced into a cell in a single reaction.

In certain embodiments, the gRNA, the nucleic acid encoding the DNA binding nuclease, the reverse transcriptase, the DNA comprising nucleic acid linked to a complementary integration site, the integration enzyme, and optionally the ngRNA are introduced using a virus, a RNP, an mRNA, a lipid, or a polymeric nanoparticle.

In certain embodiments, the nucleic acid is a reporter gene.

In certain embodiments, the reporter gene is a fluorescent protein.

In certain embodiments, the cell is a dividing cell.

In certain embodiments, the cell is a non-dividing cell.

In certain embodiments, the desired location in the cell genome is the locus of a mutated gene.

In certain embodiments, the nucleic acid is a degradation tag for programmable knockdown of proteins in the presence of small molecules.

In certain embodiments, the cell is a mammalian cell, a bacterial cell or a plant cell.

In certain embodiments, the nucleic acid is a T-cell receptor (TCR), a chimeric antigen receptor (CAR), an interleukin, a cytokine, or an immune checkpoint gene for integration into a T-cell or natural killer (NK) cell.

In certain embodiments, the TCR, the CAR, the interleukin, the cytokine, or the immune checkpoint gene is incorporated into the target site of the T-cell or NK cell genome using a minicircle DNA.

In certain embodiments, the nucleic acid is a beta hemoglobin (HBB) gene and the cell is a hematopoietic stem cell (HSC).

In certain embodiments, the HBB gene is incorporated into the target site in the HSC genome using a minicircle DNA.

In certain embodiments, the nucleic acid is a gene responsible for beta thalassemia or sickle cell anemia.

In certain embodiments, the nucleic acid is a metabolic gene.

In certain embodiments, the metabolic gene is involved in alpha-1 antitrypsin deficiency or ornithine transcarbamylase (OTC) deficiency.

In certain embodiments, the metabolic gene is a gene involved in inherited diseases.

In certain embodiments, the nucleic acid is a gene involved in an inherited disease or an inherited syndrome.

In certain embodiments, the inherited disease is cystic fibrosis, familial hypercholesterolemia, adenosine deaminase (ADA) deficiency, X-linked SCID (X-SCID), Wiskott-Aldrich syndrome (WAS), hemochromatosis, Tay-Sachs, fragile X syndrome, Huntington's disease, Marfan syndrome, phenylketonuria, muscular dystrophy, 3-methylcrotonyl-CoA carboxylase deficiency, Achromatopsia, Acute intermittent porphyria, Age related macular degeneration, Alpers-Huttenlocher syndrome, Alpha-1-antitrypsin deficiency, Alternating hemiplegia of childhood, Autosomal dominant Charcot-Marie-Tooth disease type 21, Autosomal dominant non-syndromic sensorineural deafness type DFNA, Autosomal dominant optic atrophy, classic form, Autosomal dominant progressive external ophthalmoplegia, Autosomal recessive limb-girdle muscular dystrophy type 2A, Autosomal recessive limb-girdle muscular dystrophy type 21, Bardet-Biedl syndrome, Becker muscular dystrophy, Bietti crystalline dystrophy, Bifunctional enzyme deficiency, Biotinidase deficiency, Blue cone monochromatism, Canavan disease, Charcot-Marie-Tooth disease type 1A, Charcot-Marie-Tooth disease type 1B, Charcot-Marie-Tooth disease type 2A, Choroideremia, Chronic hepatic porphyria, Citrullinemia type I, CLN1 disease, CLN4A disease, CLN4B disease, Cone rod dystrophy, Congenital glaucoma, Dentin dysplasia, Dravet syndrome, Duchenne muscular dystrophy, Ehlers-Danlos syndrome, classic type, Ehlers-Danlos syndrome, vascular type, Facioscapulohumeral dystrophy, Friedreich ataxia, Fuchs endothelial corneal dystrophy, Gaucher disease, Glutaryl-CoA dehydrogenase deficiency, Glycogen storage disease due to acid maltase deficiency, Glycogen storage disease due to liver phosphorylase kinase deficiency, Glycogen storage disease due to muscle glycogen phosphorylase deficiency, Hemolytic anemia due to red cell pyruvate kinase deficiency, Hemophilia A, Hemophilia B, Hereditary ATTR amyloidosis, Hereditary elliptocytosis, Hereditary fructose intolerance, Hereditary hemochromatosis, Hereditary hemorrhagic telangiectasia, Hereditary sensory and autonomic neuropathy type 1, Hereditary spherocytosis, Hereditary thrombophilia due to congenital antithrombin deficiency, Histidinemia, Huntington disease, Hypocalcemic vitamin D-dependent rickets, Hypokalemic periodic paralysis, Immune thrombocytopenic purpura, Infantile epileptic-dyskinetic encephalopathy, Isolated complex I deficiency, Juvenile glaucoma, Juvenile myoclonic epilepsy, Kallmann syndrome, Kennedy disease, Krabbe disease, Lattice corneal dystrophy type I, Leber congenital amaurosis, Leber hereditary optic neuropathy, Lissencephaly due to LIS1 mutation, Marfan syndrome, Medium chain acyl-CoA dehydrogenase deficiency, MERRF, Mucopolysaccharidosis type 2, Neurofibromatosis type 1, Neurofibromatosis type 2, Niemann-Pick disease type C, Oculocutaneous albinism type 1, Oculocutaneous albinism type 2, Oculocutaneous albinism type 4, Ornithine transcarbamylase deficiency, Osteogenesis imperfecta, Paramyotonia congenita of Von Eulenburg, Porphyria, Progressive familial intrahepatic cholestasis, Progressive supranuclear palsy, Proximal spinal muscular atrophy, Pseudoxanthoma elasticum, Retinitis pigmentosa, Retinitis punctata albescens, Rett syndrome, Rhizomelic chondrodysplasia punctata, Semantic dementia, Short chain acyl-CoA dehydrogenase deficiency, Sickle cell anemia, Spastic paraplegia type 7, Stargardt disease, Stickler syndrome, Thrombotic thrombocytopenic purpura, Tritanopia, Typical nemaline myopathy, Tyrosinemia type 1, Usher syndrome, Very long chain acyl-CoA dehydrogenase deficiency, Vitelliform macular dystrophy, von Gierke's disease, Von Hippel-Lindau disease, Von Willebrand disease, Waardenburg syndrome, Wilson disease, Wiskott-Aldrich syndrome, X-linked adrenoleukodystrophy.

In one aspect, the disclosure provides an integrase or fragment thereof comprising an amino acid sequence that is at least 80% identical to any one of the integrase amino acid sequences set forth in Table 8.

In certain embodiments, the integrase fragment comprises integrase, recombinase, or transposase activity.

In certain embodiments, the integrase comprises one or more mutations.

In certain embodiments, the integrase binds to nucleic acid attachment sites attB and attP, other recognition site pairs, or any pseudosites in a human genome.

In certain embodiments, the attB and/or attP nucleic acid sequence is between 12 and 60 nucleotides in length or between 18 and 50 nucleotides in length.

In certain embodiments, the attB and/or attP nucleic acid sequence comprises one or more truncations.

In certain embodiments, the attB and/or attP nucleic acid sequence is truncated by 1 to 32 nucleotides from one or both of the 5′ end and 3′ end.

In certain embodiments, the integrase binds to the corresponding attB nucleic acid sequence set forth in Table 8.

In certain embodiments, the integrase binds to the corresponding attP nucleic acid sequences set forth in Table 8.

In certain embodiments, any one of the attB nucleic acid sequences selected from the group set forth in Table 8 is truncated by 1 to 32 nucleotides from one or both of the 5′ end and 3′ end.

In certain embodiments, any one of the attP nucleic acid sequences selected from the group set forth in Table 8 is truncated by 1 to 32 nucleotides from one or both of the 5′ end and 3′ end.

In one aspect, the disclosure provides a fusion protein comprising DNA binding nuclease, a reverse transcriptase domain, and an integrase or fragment thereof, wherein the DNA binding nuclease is linked to the reverse transcriptase domain and/or the integrase or fragment thereof via a linker, wherein the integrase or fragment thereof comprises an amino acid sequence that is at least 80% identical to any one of the integrase amino acid sequences set forth in Table 8.

### BRIEF DESCRIPTION OF THE DRAWINGS

Aspects, features, benefits and advantages of the embodiments described herein will be apparent with regard to the following description, appended claims, and accompanying drawings.

FIG. 1 shows a schematic diagram of a concept of Programmable Addition via Site-Specific Targeting Elements (PASTE) according to embodiments of the present teachings.

FIG. 2 shows a schematic representation of using Bxb1 to integrate a nucleic acid into the genome according to embodiments of the present teachings.

FIG. 3 shows the percent integration of GFP or Gluc into the attB locus using Bxb1 Programmable Addition via Site-Specific Targeting Elements (PASTE) according to embodiments of the present teachings.

FIG. 4 shows the percent editing of various HEK3 targeting pegRNA Programmable Addition via Site-Specific Targeting Elements (PASTE) according to embodiments of the present teachings.

FIG. 5A-FIG. 5C shows a schematic of the integrase discovery pipeline from bacterial and metagenomic sequences (FIG. 5A) and the phylogenetic tree of discovered integrases showing distinct subfamilies (FIG. 5B and FIG. 5C).

FIG. 6A-FIG. 6I show the activity of several integrases. FIG. 6A shows an Integrase integration activity screen using reporters in HEK293FT cells compared to BxbINT and phiC31a. FIG. 6B shows PASTE integration activity with the most active integrases compared to BxbINT. FIG. 6C shows a characterization of integrase integration activity with truncated attachment sites using reporters in HEK293FT cells. FIG. 6D shows PASTE integration activity with BceINT and BcyINT with truncated attachment sites compared to BxbINT. FIG. 6E shows PASTE integration activity with SscINT and SacINT with truncated attachment sites compared to BxbINT. FIG. 6F shows optimization BceINT and SacINT PASTE constructs via protein fusions for different sized attachment sites compared to BxbINT-based PASTE for EGFP integration at the ACTB locus. FIG. 6G shows BceINT and INT2 PASTE protein constructs compared to BxbINT for EGFP integration at the ACTB locus. FIG. 6H shows integration of EGFP at different endogenous genes for PASTE with either BceINT or BxbINT. FIG. 6I shows PASTE integration activity with various integrases of EGFP at the ACTB locus.

FIG. 7A-FIG. 7B show the activity of several integrases. FIG. 7A shows an Integrase integration activity screen using reporters in HEK293FT cells compared to BxbINT, SacINTd, and BceINT. FIG. 7B shows PASTE integration activity with the integrase N352807_16_14 compared to BxbINT. N352807_16_14 was tested with various truncations of the attB sequence.

### DETAILED DESCRIPTION

It will be appreciated that for clarity, the following discussion will describe various aspects of embodiments of the applicant's teachings. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment. Furthermore, the particular feature, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an,” and “the” include both singular and plural forms unless the context clearly dictates otherwise. Thus, for example, reference to “a cell” includes a plurality of such cells.

As used herein, the term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, the term “about” or “approximately” refers to a measurable value such as a parameter, an amount, a temporal duration, and the like, are meant to encompass variations of and from the specified value, such as variations of +/−10% or less, +/−5% or less, +/−1% or less, +/−0.5% or less, and +/−0.1% or less of and from the specified value, insofar such variations are appropriate to perform in the disclosure. It is to be understood that the value to which the modifier “about” or “approximately” refers is itself also specifically disclosed.

It is noted that all publications and references cited herein are expressly incorporated herein by reference in their entirety. The publications discussed herein are provided solely for their disclosure prior to the filing date of the present application. Nothing herein is to be construed as an admission that the present disclosure is not entitled to antedate such publication. Further, the dates of publication provided may be different from the actual publication dates, which may need to be independently confirmed.

### Overview

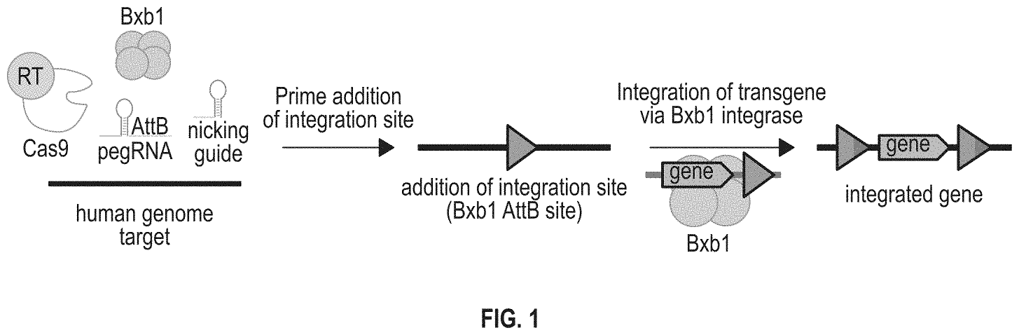

The embodiments disclosed herein provide non-naturally occurring or engineered systems, methods, and compositions for site-specific genetic engineering using Programmable Addition via Site-Specific Targeting Elements (PASTE). A schematic diagram illustrating the concept of PASTE is shown in FIG. 1. As discussed in more details below, the PASTE comprises the addition of an integration site into a target genome followed by the insertion of one or more genes of interest or one or more nucleic acid sequences of interest at the site. This process can be done as one or more reactions into a cell. The addition of the integration site into the target genome is done using gene editing technologies that include for example, without limitation, prime editing, recombinant adeno-associated virus (rAAV)-mediated nucleic acid integration, transcription activator-like effector nucleases (TALENS), and zinc finger nucleases (ZFNs). The integration of the transgene at the integration site is done using integrase technologies that include for example, without limitation, integrases, recombinases and reverse transcriptases. The necessary components for the site-specific genetic engineering disclosed herein comprise at least one or more nucleases, one or more guide RNA (gRNA), one or more integration enzymes, and one or more sequences that are complementary or associated to the integration site and linked to the one or more genes of interest or one or more nucleic acid sequences of interest to be inserted into the cell genome.

An advantage of the non-naturally occurring or engineered systems, methods, and compositions for site-specific genetic engineering disclosed herein is programmable insertion of large elements without reliance on DNA damage responses.

Another advantage of the non-naturally occurring or engineered systems, methods, and compositions for site-specific genetic engineering disclosed herein is facile multiplexing, enabling programmable insertion at multiple sites.

Yet another advantage of the non-naturally occurring or engineered systems, methods, and compositions for site-specific genetic engineering disclosed herein is scalable production and delivery through minicircle templates.

### Prime Editing

The present disclosure provides non-naturally occurring or engineered systems, methods, and compositions for site-specific genetic engineering using gene editing technologies such as prime editing to add an integration site into a target genome. Prime editing will be discussed in more detail below.

Prime editing is a versatile and precise genome editing method that directly writes new genetic information into a specified DNA site. Such method is explained fully in the literature. See, e.g., Anzalone, A. V., et al. “Search-and-replace genome editing without double-strand breaks or donor DNA,” Nature 576, 149-157 (2019). Prime editing uses a catalytically-impaired Cas9 endonuclease that is fused to an engineered reverse transcriptase (RT) and programmed with a prime-editing guide RNA (pegRNA). The skilled person in the art would appreciate that the pegRNA both specifies the target site and encodes the desired edit. The catalytically-impaired Cas9 endonuclease also comprises a Cas9 nickase that is fused to the reverse transcriptase. During genetic editing, the Cas9 nickase part of the protein is guided to the DNA target site by the pegRNA. The reverse transcriptase domain then uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Afterward, the prime editor (PE) guides resolution of the heteroduplex to favor copying the edit onto the unedited strand, completing the process.

The prime editors refer to a Moloney Murine Leukemia Virus (M-MLV) reverse transcriptase (RT) fused to a Cas9 H840A nickase. Fusing the RT to the C-terminus of the Cas9 nickase may result in higher editing efficiency. Such a complex is called PE1. The Cas9(H840A) can also be linked to a non-M-MLV reverse transcriptase such as a AMV-RT or XRT (Cas9(H840A)-AMV-RT or XRT). In some embodiments, Cas 9(H840A) can be replaced with Cas12a/b or Cas9(D10A). A Cas9 (wild type), Cas9(H840A), Cas9(D10A) or Cas 12a/b nickase fused to a pentamutant of M-MLV RT (D200N/L603W/T330P/T306K/W313F), having up to about 45-fold higher efficiency is called PE2. In some embodiments, the M-MLV RT comprise one or more of the mutations Y8H, P51L, S56A, S67R, E69K, V129P, L139P, T197A, H204R, V223H, T246E, N249D, E286R, Q291I, E302K, E302R, F309N, M320L, P330E, L435G, L435R, N454K, D524A, D524G, D524N, E562Q, D583N, H594Q, E607K, D653N, and L671P. In some embodiments, the reverse transcriptase can also be a wild-type or modified transcription xenopolymerase (RTX), avian myeloblastosis virus reverse transcriptase (AMV-RT), Feline Immunodeficiency Virus reverse transcriptase (FIV-RT), FeLV-RT (Feline leukemia virus reverse transcriptase), HIV-RT (Human Immunodeficiency Virus reverse transcriptase), or Eubacterium rectale maturase RT (MarathonRT). PE3 involves nicking the non-edited strand, potentially causing the cell to remake that strand using the edited strand as the template to induce HR. The nicking of the non-edited strand can involve the use of a nicking guide RNA (ngRNA).

In certain embodiments, the reverse transcriptase contains a stabilization domain. In certain embodiments, the stabilization domain comprises the DNA-binding Sto7d protein from Sulfolobus tokodaii or the DNA-binding Sso7d protein. The DNA-binding proteins improves processivity and resistance to inhibitors of M-MuLV reverse transcriptase. The DNA-binding Sto7d protein from Sulfolobus tokodaii or the DNA-binding Sso7d protein are described in further detail in Oscorbin et al. (FEBS Letters. 594(24): 4338-4356. 2020), incorporated herein by reference.

Nicking the non-edited strand can increase editing efficiency. For example, nicking the non-edited strand can increase editing efficiency by about 1.1 fold, about 1.3 fold, about 1.5 fold, about 1.7 fold, about 1.9 fold, about 2.1 fold, about 2.3 fold, about 2.5 fold, about 2.7 fold, about 2.9 fold, about 3.1 fold, about 3.3 fold, about 3.5 fold, about 3.7 fold, about 3.9 fold, 4.1 fold, about 4.3 fold, about 4.5 fold, about 4.7 fold, about 4.9 fold, or any range that is formed from any two of those values as endpoints.

Although the optimal nicking position varies depending on the genomic site, nicks positioned 3′ of the edit about 40-90 bp from the pegRNA-induced nick can generally increase editing efficiency without excess indel formation. The prime editing practice allows starting with non-edited strand nicks about 50 bp from the pegRNA-mediated nick, and testing alternative nick locations if indel frequencies exceed acceptable levels.

As used herein, the term “guide RNA” (gRNA) and the like refer to an RNA that guides the insertion or deletion of one or more genes of interest or one or more nucleic acid sequences of interest into a target genome. The gRNA can also refer to a prime editing guide RNA (pegRNA), a nicking guide RNA (ngRNA), and a single guide RNA (sgRNA). In some embodiments, the term “gRNA molecule” refers to a nucleic acid encoding a gRNA. In some embodiments, the gRNA molecule is naturally occurring. In some embodiments, a gRNA molecule is non-naturally occurring. In some embodiments, a gRNA molecule is a synthetic gRNA molecule. A gRNA can target a nuclease or a nickase such as Cas9, Cas 12a/b Cas9(H840A) or Cas9 (D10A) molecule to a target nucleic acid or sequence in a genome. In some embodiments, the gRNA can bind to a DNA nickase bound to a reverse transcriptase domain. A “modified gRNA,” as used herein, refers to a gRNA molecule that has an improved half-life after being introduced into a cell as compared to a non-modified gRNA molecule after being introduced into a cell. In some embodiments, the guide RNA can facilitate the addition of the insertion site sequence for recognition by integrases, transposases, or recombinases.

As used herein, the term “prime-editing guide RNA” (pegRNA) and the like refer to an extended single guide RNA (sgRNA) comprising a primer binding site (PBS), a reverse transcriptase (RT) template sequence, and an integration site sequence that can be recognized by recombinases, integrases, or transposases. For example, the PBS can have a length of at least about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, or more nt. For example, the PBS can have a length of about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, or any range that is formed from any two of those values as endpoints. For example, the RT template sequence can have a length of at least about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, or more nt. For example, the RT template sequence can have a length of about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, or any range that is formed from any two of those values as endpoints.

During genome editing, the primer binding site allows the 3′ end of the nicked DNA strand to hybridize to the pegRNA, while the RT template serves as a template for the synthesis of edited genetic information. The pegRNA is capable for instance, without limitation, of (i) identifying the target nucleotide sequence to be edited and (ii) encoding new genetic information that replaces the targeted sequence. In some embodiments, the pegRNA is capable of (i) identifying the target nucleotide sequence to be edited and (ii) encoding an integration site that replaces the targeted sequence.

As used herein, the term “nicking guide RNA” (ngRNA) and the like refer to an RNA sequence that can nick a strand such as an edited strand and a non-edited strand. The ngRNA can induce nicks at about 1 or more nt away from the site of the gRNA-induced nick. For example, the ngRNA can nick at least at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, or more nt away from the site of the gRNA induced nick. As used herein, the terms “reverse transcriptase” and “reverse transcriptase domain” refer to an enzyme or an enzymatically active domain that can reverse a RNA transcribe into a complementary DNA. The reverse transcriptase or reverse transcriptase domain is a RNA dependent DNA polymerase. Such reverse transcriptase domains encompass, but are not limited, to a M-MLV reverse transcriptase, or a modified reverse transcriptase such as, without limitation, Superscript® reverse transcriptase (Invitrogen; Carlsbad, Calif.), Superscript® VILO™ cDNA synthesis (Invitrogen; Carlsbad, Calif.), RTX, AMV-RT, and Quantiscript Reverse Transcriptase (Qiagen, Hilden, Germany).

The pegRNA-PE complex disclosed herein recognizes the target site in the genome and the Cas9 for example nicks a protospacer adjacent motif (PAM) strand. The primer binding site (PBS) in the pegRNA hybridizes to the PAM strand. The RT template operably linked to the PBS, containing the edit sequence, directs the reverse transcription of the RT template to DNA into the target site. Equilibration between the edited 3′ flap and the unedited 5′ flap, cellular 5′ flap cleavage and ligation, and DNA repair results in stably edited DNA. To optimize base editing, a Cas9 nickase can be used to nick the non-edited strand, thereby directing DNA repair to that strand, using the edited strand as a template.

### Integrase Technologies

The present disclosure provides non-naturally occurring or engineered systems, methods, and compositions for site-specific genetic engineering using integrase technologies. Integrase technologies will be discussed in more detail below.

The integrase technologies used herein comprise proteins or nucleic acids encoding the proteins that direct integration of a gene of interest or nucleic acid sequence of interest into an integration site via a nuclease such as a prime editing nuclease. In certain embodiments, the protein directing the integration can be an enzyme such as an integration enzyme. In certain embodiments, the integration enzyme can be an integrase that incorporates the genome or nucleic acid of interest into the cell genome at the integration site by integration.

As used herein, the term “integration enzyme” refers to an enzyme or protein used to integrate a gene of interest or nucleic acid sequence of interest into a desired location or at the integration site, in the genome of a cell, in a single reaction or multiple reactions. In certain embodiments, the integrase or fragment thereof comprises an amino acid sequence that is at least 80% identical to an amino acid sequence set forth in any one of SEQ ID NOs: 1-16. In certain embodiments, the integrase or fragment thereof comprises an amino acid sequence that is about 90% identical, about 91% identical, about 92% identical, about 93% identical, about 94% identical, about 95% identical, about 96% identical, about 97% identical, about 98% identical, about 99% identical, or 100% identical to an amino acid sequence set forth in any one of SEQ ID NOs: 1-16.

In certain embodiments, the integrase fragment comprises (e.g., retains) integrase activity.

In certain embodiments, the integrase further comprises one or more mutations. Mutations include, but are not limited to, amino acid substitutions, amino acid deletions, and amino acid insertions.

In some embodiments, the term “integration enzyme” refers to a nucleic acid (DNA or RNA) encoding the above-mentioned enzymes.

In some embodiments, the serine integrase φC31 from φC31 phage is used as an integration enzyme. The integrase φC31 in combination with a pegRNA can be used to insert the pseudo attP integration site (CCCCAACTGGGGTAACCTTTGAGTTCTCTCAGTTGGGG (SEQ ID NO: 54)). A DNA minicircle containing a gene or nucleic acid of interest and attB (GGCCGGCTTGTCGACGACGGCGGTCTCCGTCGTCAGGATCATCCGG (SEQ ID NO: 55)) site can be used to integrate the gene or nucleic acid of interest into the genome of a cell. This integration can be aided by a co-transfection of an expression vector having the φC31 integrase.

As used herein, the term “integrase” refers to a bacteriophage derived integrase, including wild-type integrase and any of a variety of mutant or modified integrases. As used herein, the term “integrase complex” may refer to a complex comprising integrase and integration host factor (IF). As used herein, the term “integrase complex” and the like may also refer to a complex comprising an integrase, an integration host factor, and a bacteriophage X-derived excisionase.

### Integration Site

The present disclosure provides non-naturally occurring or engineered systems, methods, and compositions for site-specific genetic engineering via the addition of an integration site into a target genome. The integration site will be discussed in more details below.

As used herein, the term “integration site” refers to the site within the target genome where one or more genes of interest or one or more nucleic acid sequences of interest are inserted.

The integration site can be inserted into the genome or a fragment thereof of a cell using a nuclease, a gRNA, and/or an integration enzyme. The integration site can be inserted into the genome of a cell using a prime editor such as, without limitation, PE1, PE2, and PE3, wherein the integration site is carried on a pegRNA. The pegRNA can target any site that is known in the art. Examples of cites targeted by the pegRNA include, without limitation, ACTB, SUPT16H, SRRM2, NOLC1, DEPDC4, NES, LMNB1, AAVS1 locus, CC10, CFTR, SERPINAL ABCA4, and any derivatives thereof. The complementary integration site may be operably linked to a gene of interest or nucleic acid sequence of interest in an exogenous DNA or RNA. In some embodiments, one integration site is added to a target genome. In some embodiments, more than one integration sites are added to a target genome.

To insert multiple genes or nucleic acids of interest, two or more integration sites are added to a desired location. Multiple DNA comprising nucleic acid sequences of interest are flanked orthogonal to the integration sequences such as, without limitation, attB, attP, other recognition site pairs, or any pseudosites in the human genome. As used herein, a “pseudosite” is a nucleic acid sequence in the target genome (e.g., a human genome) that is similar to a wild type attB or attP sequences. The sequence similarity is sufficient to allow integration of a nucleic acid sequence with an integrase enzyme. An integration site is “orthogonal” when it does not significantly recognize the recognition site or nucleotide sequence of a recombinase. Thus, one attB site of a recombinase can be orthogonal to an attB site of a different recombinase. In addition, one pair of attB and attP sites of a recombinase can be orthogonal to another pair of attB and attP sites recognized by the same recombinase. A pair of recombinases are considered orthogonal to each other, as defined herein, when there is recognition of each other's attB or attP site sequences. In certain embodiments, the attB nucleic acid sequences selected from the group consisting of SEQ ID NOs: 17, 19, 21, 23, 25, 27, 29, 31, 33, 35, 37, 39, 41, 43, 45, and 47. In certain embodiments, the attP nucleic acid sequences selected from the group consisting of SEQ ID NOs: 18, 20, 22, 24, 26, 28, 30, 32, 34, 36, 38, 40, 42, 44, 46, and 48. In certain embodiments, the attB/attP nucleic acid pair is selected from the group consisting of: SEQ ID NO: 17/SEQ ID NO: 18, SEQ ID NO: 19/SEQ ID NO: 20, SEQ ID NO: 21/SEQ ID NO: 22, SEQ ID NO: 23/SEQ ID NO: 24, SEQ ID NO: 25/SEQ ID NO: 26, SEQ ID NO: 27/SEQ ID NO: 28, SEQ ID NO: 29/SEQ ID NO: 30, SEQ ID NO: 31/SEQ ID NO: 32, SEQ ID NO: 33/SEQ ID NO: 34, SEQ ID NO: 35/SEQ ID NO: 36, SEQ ID NO: 37/SEQ ID NO: 38, SEQ ID NO: 39/SEQ ID NO: 40, SEQ ID NO: 41/SEQ ID NO: 42, SEQ ID NO: 43/SEQ ID NO: 44, SEQ ID NO: 45/SEQ ID NO: 46, and SEQ ID NO: 47/SEQ ID NO: 48.

In certain embodiments, the attB nucleic acid sequence is between 12 and 60 nucleotides in length or between 18 and 50 nucleotides in length. In certain embodiments, the attB nucleic acid sequence is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length.

In certain embodiments, the attP nucleic acid sequence is between 12 and 60 nucleotides in length or between 18 and 50 nucleotides in length. In certain embodiments, the attP nucleic acid sequence is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length.

In certain embodiments, the attB and/or attP nucleic acid sequence comprises one or more truncations. The truncation may be at the 5′ end, 3′end, or both. The truncations to the attB and/or attP nucleic acids sequences may be made while still retaining the ability to bind an integrase.

In certain embodiments, the attB and/or attP nucleic acid sequence is truncated by 1 to 32 nucleotides from one or both of the 5′ end and 3′ end. In certain embodiments, the attB nucleic acid sequence is truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 nucleotides from one or both of the 5′ end and 3′ end. In certain embodiments, the attP nucleic acid sequence is truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 nucleotides from one or both of the 5′ end and 3′ end.

In certain embodiments, any one of the attB nucleic acid sequences selected from the group consisting of SEQ ID NOs: 17, 19, 21, 23, 25, 27, 29, 31, 33, 35, 37, 39, 41, 43, 45, and 47 is truncated by 1 to 32 nucleotides from one or both of the 5′ end and 3′ end. In certain embodiments, any one of the attP nucleic acid sequences selected from the group consisting of SEQ ID NOs: 18, 20, 22, 24, 26, 28, 30, 32, 34, 36, 38, 40, 42, 44, 46, and 48 is truncated by 1 to 32 nucleotides from one or both of the 5′ end and 3′ end.

The lack of recognition of integration sites can be less than about 30%. In some embodiments, the lack of recognition of integration sites or pairs of sites can be less than about 30%, less than about 28%, less than about 26%, less than about 24%, less than about 22%, less than about 20%, less than about 18%, less than about 16%, less than about 14%, less than about 12%, less than about 10%, less than about 8%, less than about 6%, less than about 4%, less than about 2%, about 1%, or any range that is formed from any two of those values as endpoints. The crosstalk can be less than about 30%. In some embodiments, the crosstalk is less than about 30%, less than about 28%, less than about 26%, less than about 24%, less than about 22%, less than about 20%, less than about 18%, less than about 16%, less than about 14%, less than about 12%, less than about 10%, less than about 8%, less than about 6%, less than about 4%, less than about 2%, less than about 1%, or any range that is formed from any two of those values as endpoints.

In some embodiments, the attB and/or attP site sequences comprise a central dinucleotide sequence. It has been shown that, for example, the central dinucleotide can be changed to GA from GT and that only GA containing attB/attP sites interact and will not cross react with GT containing sequences. In some embodiments, the central dinucleotide is selected from the group consisting of AG, AC, TG, TC, CA, CT, GA, AA, TT, CC, GG, AT, TA, GC, CG and GT.

As used herein, the term “pair of an attB and attP site sequences” and the like refer to attB and attP site sequences that share the same central dinucleotide and can recombine. This means that in the presence of one serine integrase as many as six pairs of these orthogonal att sites can recombine (attPTT will specifically recombine with attBTT, attPTC will specifically recombine with attBTC, and so on).

In some embodiments, the central dinucleotide is nonpalindromic. In some embodiments, the central dinucleotide is palindromic. In some embodiments, a pair of an attB site sequence and an attP site sequence are used in different DNA encoding genes of interest or nucleic acid sequences of interest for inducing directional integration of two or more different nucleic acids. In some embodiments, two integrases can be used for orthogonal insertion.

The Table 1 below shows examples of pairs of attB site sequence and attP site sequence with different central dinucleotide (CD).

TABLE 1

| Pair | attB | attP | CD |
|---|---|---|---|
| 1 | GGCTTGTCGACGACGGCGTTCTC | GTGGTTTGTCTGGTCAA | TT |
| | CGTCGTCAGGATCAT | CCACCGCGTTCTCAGTG | |
| | (SEQ ID NO: 56) | GTGTACGGTACAAACCC | |
| | | A (SEQ ID NO: 72) | |
| 2 | GGCTTGTCGACGACGGCGAACTC | GTGGTTTGTCTGGTCAA | AA |
| | CGTCGTCAGGATCAT | CCACCGCGAACTCAGTG | |
| | (SEQ ID NO: 57) | GTGTACGGTACAAACCC | |
| | | A (SEQ ID NO: 73) | |
| 3 | GGCTTGTCGACGACGGCGCCCTC | GTGGTTTGTCTGGTCAA | CC |
| | CGTCGTCAGGATCAT | CCACCGCGCCCTCAGTG | |
| | (SEQ ID NO: 58) | GTGTACGGTACAAACCC | |
| | | A (SEQ ID NO: 74) | |
| 4 | GGCTTGTCGACGACGGCGGGCTC | GTGGTTTGTCTGGTCAA | GG |
| | CGTCGTCAGGATCAT | CCACCGCGGGCTCAGTG | |
| | (SEQ ID NO: 59) | GTGTACGGTACAAACCC | |
| | | A (SEQ ID NO: 75) | |
| 5 | GGCTTGTCGACGACGGCGTGCTC | GTGGTTTGTCTGGTCAA | TG |
| | CGTCGTCAGGATCAT | CCACCGCGTGCTCAGTG | |
| | (SEQ ID NO: 60) | GTGTACGGTACAAACCC | |
| | | A (SEQ ID NO: 76) | |
| 6 | GGCTTGTCGACGACGGCGGTCTC | GTGGTTTGTCTGGTCAA | GT |
| | CGTCGTCAGGATCAT | CCACCGCGGTCTCAGTG | |
| | (SEQ ID NO: 61) | GTGTACGGTACAAACCC | |
| | | A (SEQ ID NO: 77) | |
| 7 | GGCTTGTCGACGACGGCGCTCTC | GTGGTTTGTCTGGTCAA | CT |
| | CGTCGTCAGGATCAT | CCACCGCGCTCTCAGTG | |
| | (SEQ ID NO: 62) | GTGTACGGTACAAACCC | |
| | | A (SEQ ID NO: 78) | |
| 8 | GGCTTGTCGACGACGGCGCACTC | GTGGTTTGTCTGGTCAA | CA |
| | CGTCGTCAGGATCAT | CCACCGCGCACTCAGTG | |
| | (SEQ ID NO: 63) | GTGTACGGTACAAACCC | |
| | | A (SEQ ID NO: 79) | |
| 9 | GGCTTGTCGACGACGGCGTCCTC | GTGGTTTGTCTGGTCAA | TC |
| | CGTCGTCAGGATCAT | CCACCGCGTCCTCAGTG | |
| | (SEQ ID NO: 64) | GTGTACGGTACAAACCC | |
| | | A (SEQ ID NO: 80) | |
| 10 | GGCTTGTCGACGACGGCGGACTC | GTGGTTTGTCTGGTCAA | GA |
| | CGTCGTCAGGATCAT | CCACCGCGGACTCAGTG | |
| | (SEQ ID NO: 65) | GTGTACGGTACAAACCC | |
| | | A (SEQ ID NO: 81) | |
| 11 | GGCTTGTCGACGACGGCGAGCTC | GTGGTTTGTCTGGTCAA | AG |
| | CGTCGTCAGGATCAT | CCACCGCGAGCTCAGTG | |
| | (SEQ ID NO: 66) | GTGTACGGTACAAACCC | |
| | | A (SEQ ID NO: 82) | |
| 12 | GGCTTGTCGACGACGGCGACCTC | GTGGTTTGTCTGGTCAA | AC |
| | CGTCGTCAGGATCAT | CCACCGCGACCTCAGTG | |
| | (SEQ ID NO: 67) | GTGTACGGTACAAACCC | |
| | | A (SEQ ID NO: 83) | |
| 13 | GGCTTGTCGACGACGGCGATCTC | GTGGTTTGTCTGGTCAA | AT |
| | CGTCGTCAGGATCAT | CCACCGCGATCTCAGTG | |
| | (SEQ ID NO: 68) | GTGTACGGTACAAACCC | |
| | | A (SEQ ID NO: 84) | |
| 14 | GGCTTGTCGACGACGGCGGCCTC | GTGGTTTGTCTGGTCAA | GC |
| | CGTCGTCAGGATCAT | CCACCGCGGCCTCAGTG | |
| | (SEQ ID NO: 69) | GTGTACGGTACAAACCC | |
| | | A (SEQ ID NO: 85) | |
| 15 | GGCTTGTCGACGACGGCGCGCTC | GTGGTTTGTCTGGTCAA | CG |
| | CGTCGTCAGGATCAT | CCACCGCGCGCTCAGTG | |
| | (SEQ ID NO: 70) | GTGTACGGTACAAACCC | |
| | | A (SEQ ID NO: 86) | |
| 16 | GGCTTGTCGACGACGGCGTACTC | GTGGTTTGTCTGGTCAA | TA |
| | CGTCGTCAGGATCAT | CCACCGCGTACTCAGTG | |
| | (SEQ ID NO: 71) | GTGTACGGTACAAACCC | |
| | | A (SEQ ID NO: 87) | |

In one aspect, the disclosure provides an integrase or fragment thereof, wherein:
a) the integrase or fragment thereof comprises an amino acid sequence that is at least 80% identical to an amino acid sequence set forth in SEQ ID NO: 1, wherein the integrase binds to the attB nucleic acid set forth in SEQ ID NO: 17 and the attP nucleic acid set forth in SEQ ID NO: 18;
b) the integrase or fragment thereof comprises an amino acid sequence that is at least 80% identical to an amino acid sequence set forth in SEQ ID NO: 2, wherein the integrase binds to the attB nucleic acid set forth in SEQ ID NO: 19 and the attP nucleic acid set forth in SEQ ID NO: 20;
c) the integrase or fragment thereof comprises an amino acid sequence that is at least 80% identical to an amino acid sequence set forth in SEQ ID NO: 3, wherein the integrase binds to the attB nucleic acid set forth in SEQ ID NO: 21 and the attP nucleic acid set forth in SEQ ID NO: 22;
d) the integrase or fragment thereof comprises an amino acid sequence that is at least 80% identical to an amino acid sequence set forth in SEQ ID NO: 4, wherein the integrase binds to the attB nucleic acid set forth in SEQ ID NO: 23 and the attP nucleic acid set forth in SEQ ID NO: 24;
e) the integrase or fragment thereof comprises an amino acid sequence that is at least 80% identical to an amino acid sequence set forth in SEQ ID NO: 5, wherein the integrase binds to the attB nucleic acid set forth in SEQ ID NO: 25 and the attP nucleic acid set forth in SEQ ID NO: 26;
f) the integrase or fragment thereof comprises an amino acid sequence that is at least 80% identical to an amino acid sequence set forth in SEQ ID NO: 6, wherein the integrase binds to the attB nucleic acid set forth in SEQ ID NO: 27 and the attP nucleic acid set forth in SEQ ID NO: 28;
g) the integrase or fragment thereof comprises an amino acid sequence that is at least 80% identical to an amino acid sequence set forth in SEQ ID NO: 7, wherein the integrase binds to the attB nucleic acid set forth in SEQ ID NO: 29 and the attP nucleic acid set forth in SEQ ID NO: 30;
h) the integrase or fragment thereof comprises an amino acid sequence that is at least 80% identical to an amino acid sequence set forth in SEQ ID NO: 8, wherein the integrase binds to the attB nucleic acid set forth in SEQ ID NO: 31 and the attP nucleic acid set forth in SEQ ID NO: 32;
i) the integrase or fragment thereof comprises an amino acid sequence that is at least 80% identical to an amino acid sequence set forth in SEQ ID NO: 9, wherein the integrase binds to the attB nucleic acid set forth in SEQ ID NO: 33 and the attP nucleic acid set forth in SEQ ID NO: 34;
j) the integrase or fragment thereof comprises an amino acid sequence that is at least 80% identical to an amino acid sequence set forth in SEQ ID NO: 10, wherein the integrase binds to the attB nucleic acid set forth in SEQ ID NO: 35 and the attP nucleic acid set forth in SEQ ID NO: 36;
k) the integrase or fragment thereof comprises an amino acid sequence that is at least 80% identical to an amino acid sequence set forth in SEQ ID NO: 11, wherein the integrase binds to the attB nucleic acid set forth in SEQ ID NO: 37 and the attP nucleic acid set forth in SEQ ID NO: 38;
l) the integrase or fragment thereof comprises an amino acid sequence that is at least 80% identical to an amino acid sequence set forth in SEQ ID NO: 12, wherein the integrase binds to the attB nucleic acid set forth in SEQ ID NO: 39 and the attP nucleic acid set forth in SEQ ID NO: 40;
m) the integrase or fragment thereof comprises an amino acid sequence that is at least 80% identical to an amino acid sequence set forth in SEQ ID NO: 13, wherein the integrase binds to the attB nucleic acid set forth in SEQ ID NO: 41 and the attP nucleic acid set forth in SEQ ID NO: 42;
n) the integrase or fragment thereof comprises an amino acid sequence that is at least 80% identical to an amino acid sequence set forth in SEQ ID NO: 14, wherein the integrase binds to the attB nucleic acid set forth in SEQ ID NO: 43 and the attP nucleic acid set forth in SEQ ID NO: 44;
o) the integrase or fragment thereof comprises an amino acid sequence that is at least 80% identical to an amino acid sequence set forth in SEQ ID NO: 15, wherein the integrase binds to the attB nucleic acid set forth in SEQ ID NO: 45 and the attP nucleic acid set forth in SEQ ID NO: 46; or
p) the integrase or fragment thereof comprises an amino acid sequence that is at least 80% identical to an amino acid sequence set forth in SEQ ID NO: 16, wherein the integrase binds to the attB nucleic acid set forth in SEQ ID NO: 47 and the attP nucleic acid set forth in SEQ ID NO: 48.

### Paste

The present disclosure provides non-naturally occurring or engineered systems, methods, and compositions for site-specific genetic engineering using PASTE. PASTE will be discussed in more details below. The PASTE system is described in greater detail in U.S. Provisional Patent Application Ser. No. 63/094,803, filed Oct. 21, 2020, and U.S. Provisional Patent Application Ser. No. 63/222,550, filed Jul. 16, 2021, each of which is incorporated herein by reference.

The site-specific genetic engineering disclosed herein is for the insertion of one or more genes of interest or one or more nucleic acid sequences of interest into a genome of a cell. In some embodiments, the gene of interest is a mutated gene implicated in a genetic disease such as, without limitation, a metabolic disease, cystic fibrosis, muscular dystrophy, hemochromatosis, Tay-Sachs, Huntington disease, Congenital Deafness, Sickle cell anemia, Familial hypercholesterolemia, adenosine deaminase (ADA) deficiency, X-linked SCID (X-SCID), and Wiskott-Aldrich syndrome (WAS). In some embodiments, the gene of interest or nucleic acid sequence of interest can be a reporter gene upstream or downstream of a gene for genetic analyses such as, without limitation, for determining the expression of a gene. In some embodiments, the reporter gene is a GFP template or a Gaussia Luciferase (G-Luciferase) template. In some embodiments, the gene of interest or nucleic acid sequence of interest can be used in plant genetics to insert genes to enhance drought tolerance, weather hardiness, and increased yield and herbicide resistance in plants. In some embodiments, the gene of interest or nucleic acid sequence of interest can be used for site-specific insertion of a protein (e.g., a lysosomal enzyme), a blood factor (e.g., Factor I, II, V, VII, X, XI, XII or XIII), a membrane protein, an exon, an intracellular protein (e.g., a cytoplasmic protein, a nuclear protein, an organellar protein such as a mitochondrial protein or lysosomal protein), an extracellular protein, a structural protein, a signaling protein, a regulatory protein, a transport protein, a sensory protein, a motor protein, a defense protein, or a storage protein, an anti-inflammatory signaling molecules into cells for treatment of immune diseases, including but not limited to arthritis, psoriasis, lupus, coeliac disease, glomerulonephritis, hepatitis, and inflammatory bowel disease.

The size of the inserted gene or nucleic acid can vary from about 1 bp to about 50,000 bp. In some embodiments, the size of the inserted gene or nucleic acid can be about 1 bp, 10 bp, 50 bp, 100 bp, 150 bp, 200 bp, 250 bp, 300 bp, 350 bp, 400 bp, 600 bp, 800 bp, 1000 bp, 1200 bp, 1400 bp, 1600 bp, 1800 bp, 2000 bp, 2200 bp, 2400 bp, 2600 bp, 2800 bp, 3000 bp, 3200 bp, 3400 bp, 3600 bp, 3800 bp, 4000 bp, 4200 bp, 4400 bp, 4600 bp, 4800 bp, 5000 bp, 5200 bp, 5400 bp, 5600 bp, 5800 bp, 6000 bp, 6200, 6400 bp, 6600 bp, 6800 bp, 7000 bp, 7200 bp, 7400 bp, 7600 bp, 7800 bp, 8000 bp, 8200 bp, 8400 bp, 8600 bp, 8800 bp, 9000 bp, 9200 bp, 9400 bp, 9600 bp, 9800 bp, 10,000 bp, 10,200 bp, 10,400 bp, 10,600 bp, 10,800 bp, 11,000 bp, 11,200 bp, 11,400 bp, 11,600 bp, 11,800 bp, 12,000 bp, 14,000 bp, 16,000 bp, 18,000 bp, 20,000 bp, 30,000 bp, 40,000 bp, 50,000 bp, or any range that is formed from any two of those values as endpoints.

In some embodiments, the site-specific engineering using the gene of interest or nucleic acid sequence of interest disclosed herein is for the engineering of T cells and NKs for tumor targeting or allogeneic generation. These can involve the use of receptor or CAR for tumor specificity, anti-PD1 antibody, cytokines like IFN-gamma, TNF-alpha, IL-15, IL-12, IL-18, IL-21, and IL-10, and immune escape genes.

In the present disclosure, the site-specific insertion of the gene of interest or nucleic acid of interest is performed through Programmable Addition via Site-Specific Targeting Elements (PASTE). Components for inserting a gene of interest or a nucleic acid of interest using PASTE are for example, without limitation, a nuclease, a gRNA adding the integration site, a DNA or RNA strand comprising the gene or nucleic acid linked to a sequence that is complementary or associated to the integration site, and an integration enzyme. Components for inserting a gene of interest or a nucleic acid of interest using PASTE are for example, without limitation, a prime editor expression, pegRNA adding the integration site, nicking guide RNA, integration enzyme (an integrase, such as an integrase of any one of SEQ ID NOs: 1-16), transgene vector comprising the gene of interest or nucleic acid sequence of interest with gene and integration signal. The nuclease and prime editor integrate the integration site into the genome. The integration enzyme integrates the gene of interest into the integration site. In some embodiments, the transgene vector comprising the gene or nucleic acid sequence of interest with gene and integration signal is a DNA minicircle devoid of bacterial DNA sequences. In some embodiments, the transgenic vector is a eukaryotic or prokaryotic vector.

As used herein, the term “vector” or “transgene vector” refers to a recombinant DNA molecule containing a desired coding sequence and appropriate nucleic acid sequences necessary for the expression of the operably linked coding sequence in a host organism. Nucleic acid sequences necessary for expression in prokaryotes usually include for example, without limitation, a promoter, an operator (optional), a ribosome binding site, and/or other sequences. Eukaryotic cells are generally known to utilize promoters (constitutive, inducible or tissue specific), enhancers, and termination and polyadenylation signals, although some elements may be deleted and other elements added without sacrificing the necessary expression. The transgenic vector may encode the PE and the integration enzyme, linked to each other via a linker. The linker can be a cleavable linker. In some embodiments, the linker can be a non-cleavable linker. In some embodiments the nuclease, prime editor, and/or integration enzyme can be encoded in different vectors.

In one aspect, the disclosure provides a method of inserting multiple genes or nucleic acid sequences of interest into a single site. In some embodiments, multiplexing involves inserting multiple genes of interest in multiple loci using unique pegRNA (Merrick, C. A. et al., ACS Synth. Biol. 2018, 7, 299-310). The insertion of multiple genes of interest or nucleic acids of interest into a cell genome, referred herein as “multiplexing,” is facilitated by incorporation of the complementary 5′ integration site to the 5′ end of the DNA or RNA comprising the first nucleic acid and 3′ integration site to the 3′ end of the DNA or RNA comprising the last nucleic acid. In some embodiments, the number of genome of interest or amino acid sequences of interest that are inserted into a cell genome using multiplexing can be about 1, 2, 3, 4, 5, 6, 7, 8, 9 10, 11, 12, 13, 14, 15, 16, 17, 18, 19, 20, 21, 22, 23, 24, 25, or any range that is formed from any two of those values as endpoints.

In some embodiments, multiplexing allows integration of for example, signaling cascade, over-expression of a protein of interest with its cofactor, insertion of multiple genes mutated in a neoplastic condition, or insertion of multiple CARs for treatment of cancer.

In some embodiments, the integration sites may be inserted into the genome using non-prime editing methods such as rAAV mediated nucleic acid integration, TALENS and ZFNs. A number of unique properties make AAV a promising vector for human gene therapy (Muzyczka, CURRENT TOPICS IN MICROBIOLOGY AND IMMUNOLOGY, 158:97-129 (1992)). Unlike other viral vectors, AAVs have not been shown to be associated with any known human disease and are generally not considered pathogenic. Wild type AAV is capable of integrating into host chromosomes in a site-specific manner M. Kotin et al., PROC. NATL. ACAD. SCI, USA, 87:2211-2215 (1990); R. J. Samulski, EMBO 10(12):3941-3950 (1991)). Instead of creating a double-stranded DNA break, AAV stimulates endogenous homologous recombination to achieve the DNA modification. Further, transcription activator-like effector nucleases (TALENs) and Zinc-finger nucleases (ZFNs) for genome editing and introducing targeted DSBs. The specificity of TALENs arises from two polymorphic amino acids, the so-called repeat variable diresidues (RVDs) located at positions 12 and 13 of a repeated unit. TALENS are linked to FokI nucleases, which cleaves the DNA at the desired locations. ZFNs are artificial restriction enzymes for custom site-specific genome editing. Zinc fingers themselves are transcription factors, where each finger recognizes 3-4 bases. By mixing and matching these finger modules, researchers can customize which sequence to target.

As used herein, the terms “administration,” “introducing,” or “delivery” into a cell, a tissue, or an organ of a plasmid, nucleic acids, or proteins for modification of the host genome refers to the transport for such administration, introduction, or delivery that can occur in vivo, in vitro, or ex vivo. Plasmids, DNA, or RNA for genetic modification can be introduced into cells by transfection, which is typically accomplished by chemical means (e.g., calcium phosphate transfection, polyethyleneimine (PEI) Or lipofection), physical means (electroporation or microinjection), infection (this typically means the introduction of an infectious agent such as a virus (e.g., a baculovirus expressing the AAV Rep gene)), transduction (in microbiology, this refers to the stable infection of cells by viruses, or the transfer of genetic material from one microorganism to another by viral factors (e.g., bacteriophages)). Vectors for the expression of a recombinant polypeptide, protein or oligonucleotide may be obtained by physical means (e.g., calcium phosphate transfection, electroporation, microinjection, or lipofection) in a cell, a tissue, an organ or a subject. The vector can be delivered by preparing the vector in a pharmaceutically acceptable carrier for the in vitro, ex vivo, or in vivo delivery to the carrier.

As used herein, the term “transfection” refers to the uptake of an exogenous nucleic acid molecule by a cell. A cell is “transfected” when an exogenous nucleic acid has been introduced into the cell membrane. The transfection can be a single transfection, co-transfection, or multiple transfection. Numerous transfection techniques are generally known in the art. See, for example, Graham et al. (1973) Virology, 52: 456. Such techniques can be used to introduce one or more exogenous nucleic acid molecules into a suitable host cell.

In some embodiments, the exogenous nucleic acid molecule and/or other components for gene editing are combined and delivered in a single transfection. In other embodiments, the exogenous nucleic acid molecule and/or other components for gene editing are not combined and delivered in a single transfection. In some embodiments, exogenous nucleic acid molecule and/or other components for gene editing are combined and delivered in a single transfection to comprise for example, without limitation, a prime editing vector, a landing site such as a landing site containing pegRNA, a nicking guide such as a nicking guide for stimulating prime editing, an expression vector such as an expression vector for a corresponding integrase or recombinase, a minicircle DNA cargo such as a minicircle DNA cargo encoding for green fluorescent protein (GFP), any derivatives thereof, and any combinations thereof. In some embodiments, the gene of interest or amino acid sequence of interest can be introduced using liposomes. In some embodiments, the gene of interest or amino acid sequence of interest can be delivered using suitable vectors for instance, without limitation, plasmids and viral vectors. Examples of viral vectors include, without limitation, adeno-associated viruses (AAV), lentiviruses, adenoviruses, other viral vectors, derivatives thereof, or combinations thereof. The proteins and one or more guide RNAs can be packaged into one or more vectors, e.g., plasmids or viral vectors. In some embodiments, the delivery is via nanoparticles or exosomes. For example, exosomes can be particularly useful in delivery RNA.

In some embodiments, the prime editing inserts the landing site with efficiencies of at least about 1%, at least about 5%, at least about 10%, at least about 15%, at least about, at least about 20%, at least about 25%, at least about 30%, at least about 35%, at least about 40%, at least about 45%, or at least about 50%. In some embodiments, the prime editing inserts the landing site(s) with efficiencies of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, or any range that is formed from any two of those values as endpoints.

### Sequences

Sequences of enzymes, guides, integration sites, and plasmids can be found in the Tables below.

Lengthy table referenced here
US20230272435A1-20230831-T00001
Please refer to the end of the specification for access instructions.

Lengthy table referenced here
US20230272435A1-20230831-T00002
Please refer to the end of the specification for access instructions.

Lengthy table referenced here
US20230272435A1-20230831-T00003
Please refer to the end of the specification for access instructions.

Lengthy table referenced here
US20230272435A1-20230831-T00004
Please refer to the end of the specification for access instructions.

Lengthy table referenced here
US20230272435A1-20230831-T00005
Please refer to the end of the specification for access instructions.

Lengthy table referenced here
US20230272435A1-20230831-T00006
Please refer to the end of the specification for access instructions.

Lengthy table referenced here
US20230272435A1-20230831-T00007
Please refer to the end of the specification for access instructions.

### EXAMPLES

While several experimental Examples are contemplated, these Examples are intended to be non-limiting.

### Example 1

### Bxb1 Integration Data Lenti Reporter

The PASTE system, including the description in Example 1 and Example 2, are described in greater detail in U.S. Provisional Patent Application Ser. No. 63/094,803, filed Oct. 21, 2020, and U.S. Provisional Patent Application Ser. No. 63/222,550, filed Jul. 16, 2021, each of which is incorporated herein by reference.

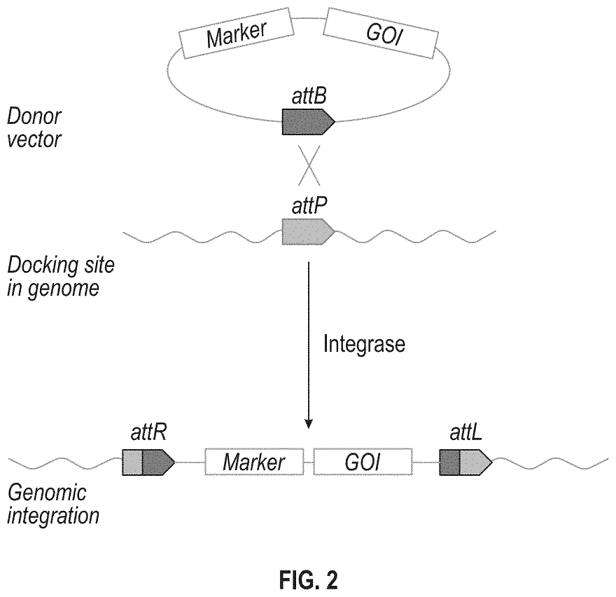

Serine integrase Bxb1 has been shown to be more active than Cre recombinase and highly efficient in bacteria and mammalian cells for irreversible integration of target genes. FIG. 1 and FIG. 2 show schematics of PASTE methodology using Bxb1 (Merrick, C. A. et al., ACS Synth. Biol. 2018, 7, 299-310).

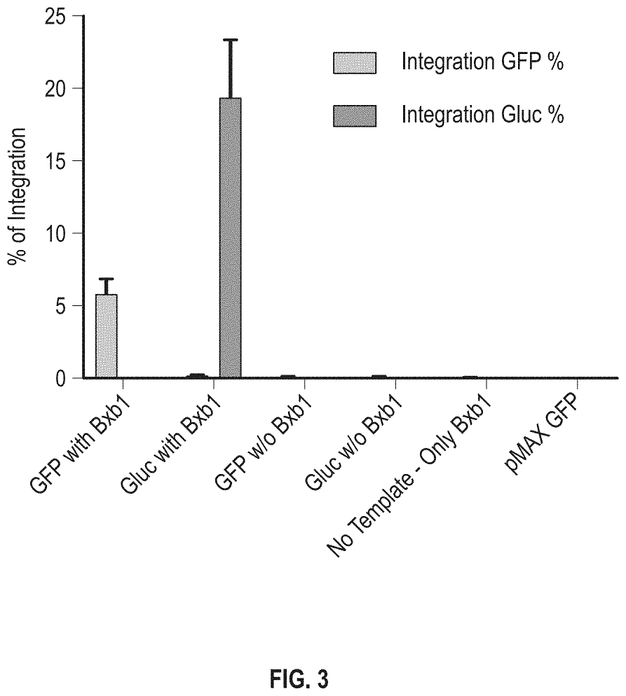

To probe the efficiency of the Bxb1 integration system, a clonal HEK293FT cell line with attB Bxb1 site (GGCCGGCTTGTCGACGACGGCGGTCTCCGTCGTCAGGATCATCCGG (SEQ ID NO: 3163)) integrated using lentivirus was developed. The modified HEK293FT cell line was then transferred with the following plasmids: (1) plus/minus Bxb1 expression plasmid and (2) plus/minus GFP or G-Luc minicircle template with attP Bxb1 site. After 72 hours, the integration of GFP or Gluc into the attB site in the HEK293FT genome was probed. The percent integrations of GFP or Gluc into the attB locus are shown in FIG. 3. It was observed that GFP and Gluc showed efficient integration into the attB site in HEK293FT cells.

### Example 2

### Addition of Bxb1 Site to Human Genome Using PRIME

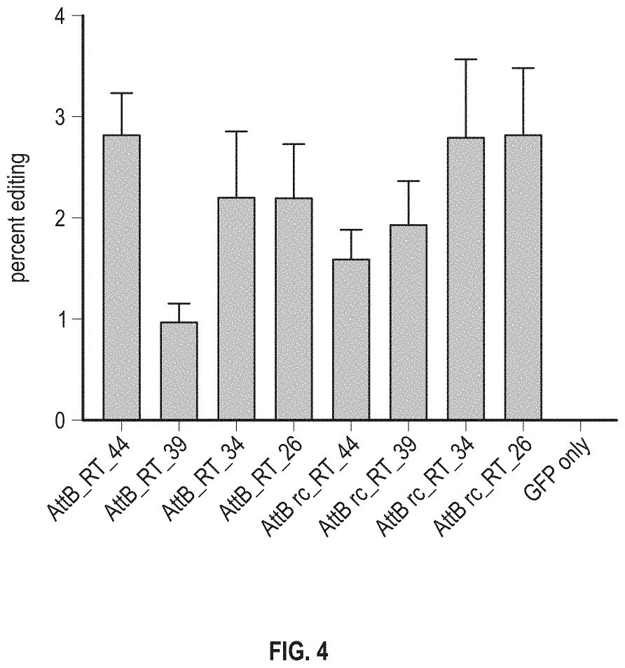

The maximum length of attB that can be integrated into a HEK293FT cell line with the best efficiency was probed. To probe the best length of attB (GGCCGGCTTGTCGACGACGGCGGTCTCCGTCGTCAGGATCATCCGG (SEQ ID NO: 3164)) or its reverse complement attP (CCGGATGATCCTGACGACGGAGACCGCCGTCGTCGACAAGCCGGCC (SEQ ID NO: 3165)) for prime editing, pegRNAs having PBS length of 13 nt with varying RT homology length were used. The following plasmids were transfected in HEK293FT: (1) prime expression plasmid; (2) HEK3 targeting pegRNA design; and (3) HEK3+90 nicking guide. After 72 hours, the percent integration of each of the attB construct was probed. FIG. 4 shows the percent editing in each HEK3 targeting pegRNA. It was observed that attB with 44, 34 and 26 base pairs and attB reverse complement with 34 and 26 base pairs showed the highest percent editing.

### Example 3

### Integrase Discovery Platform & Use in PASTE System

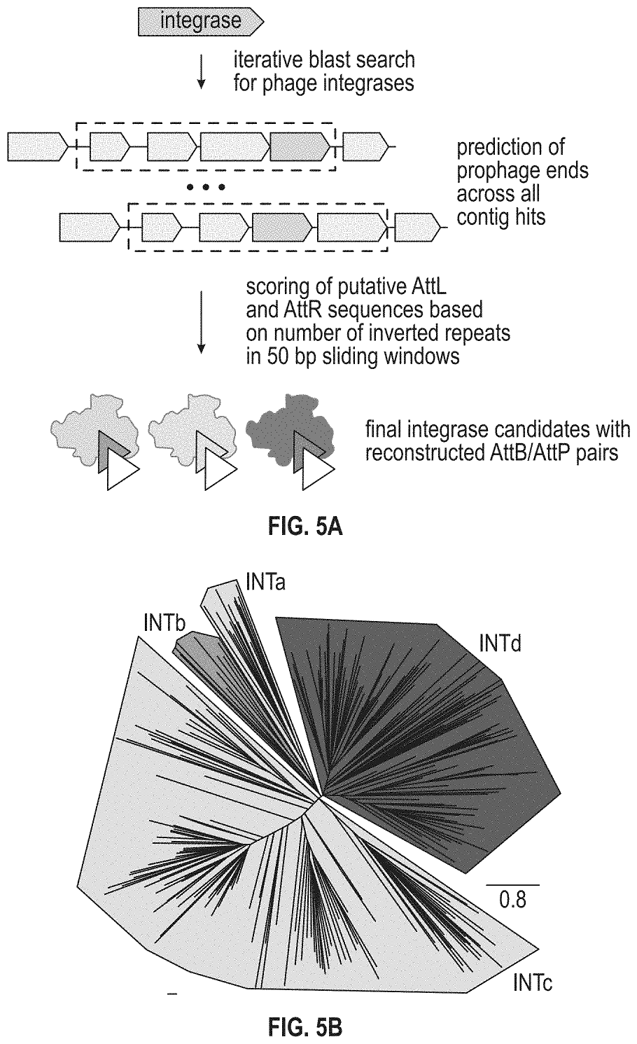

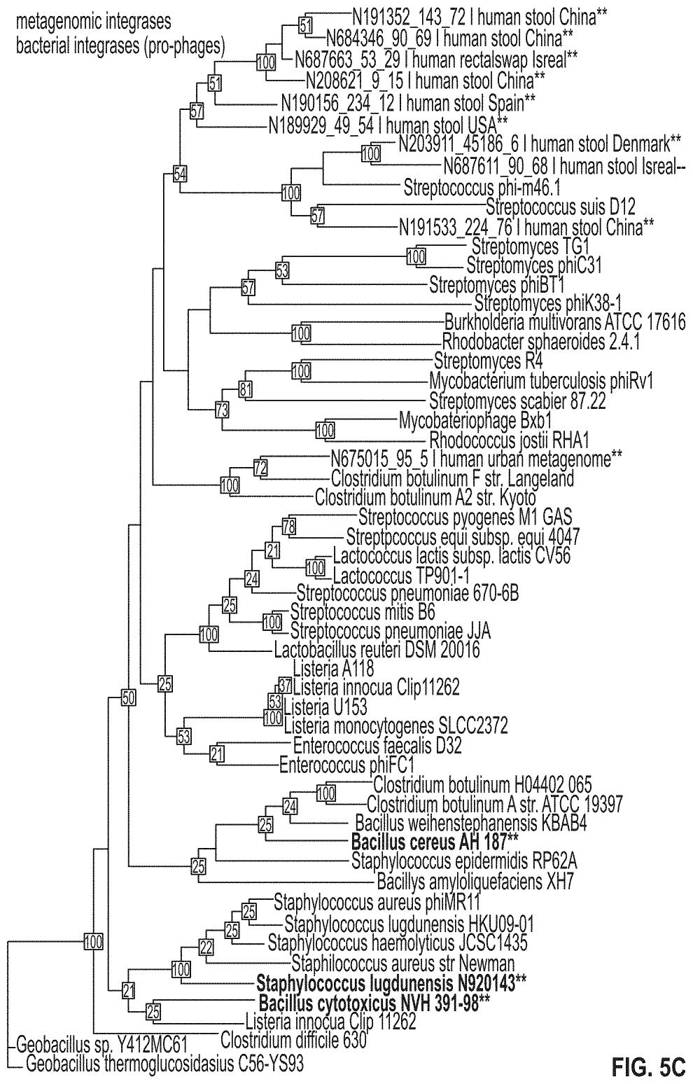

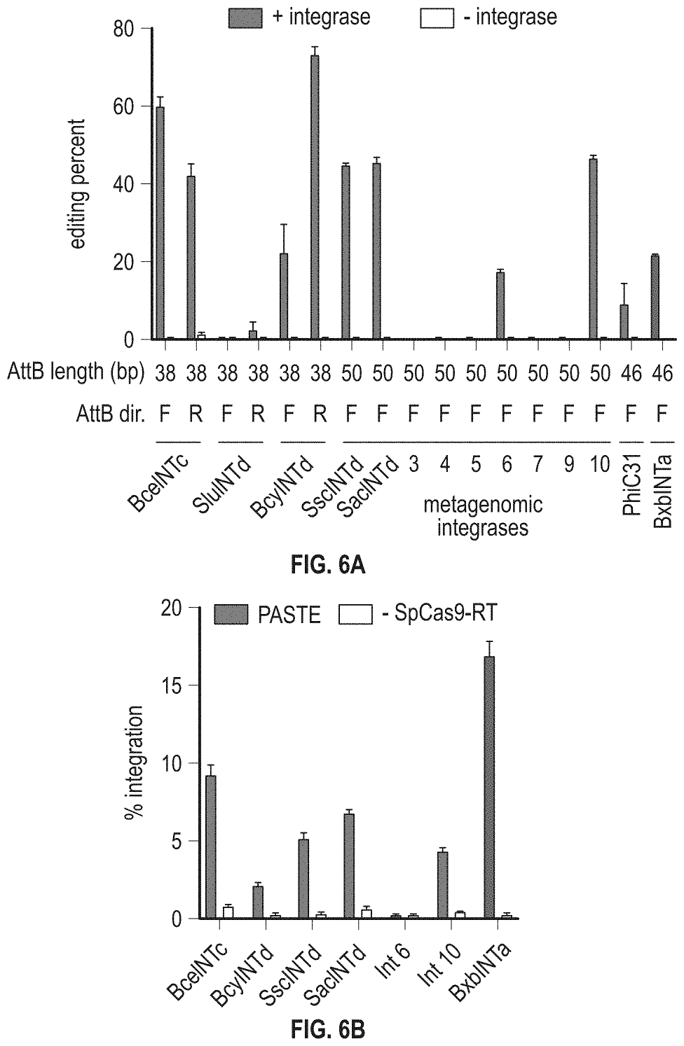

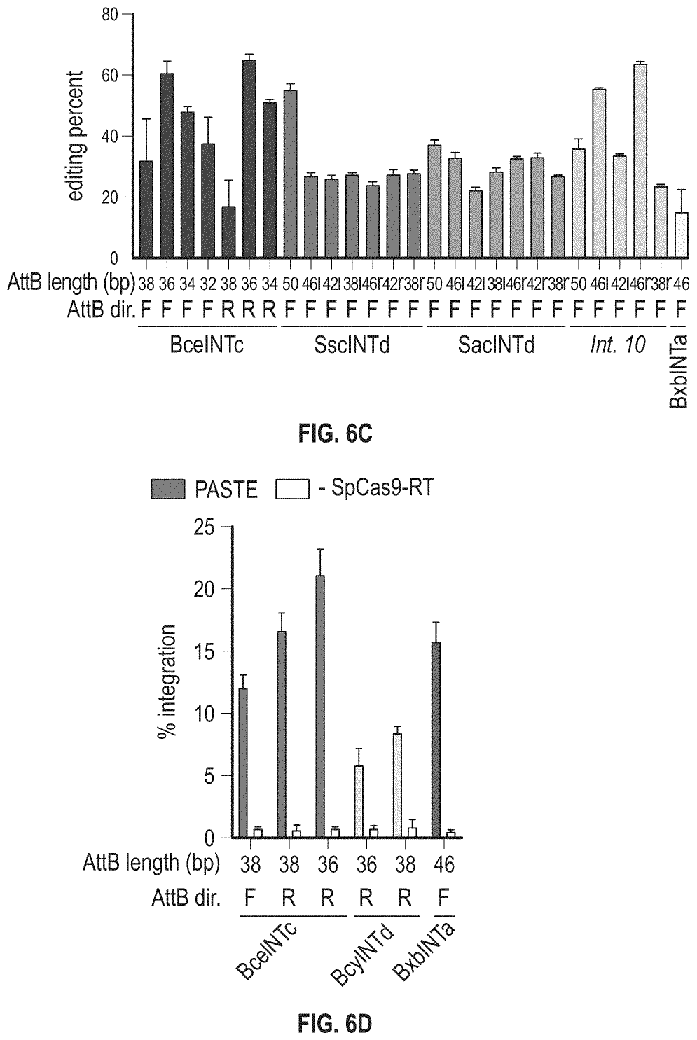

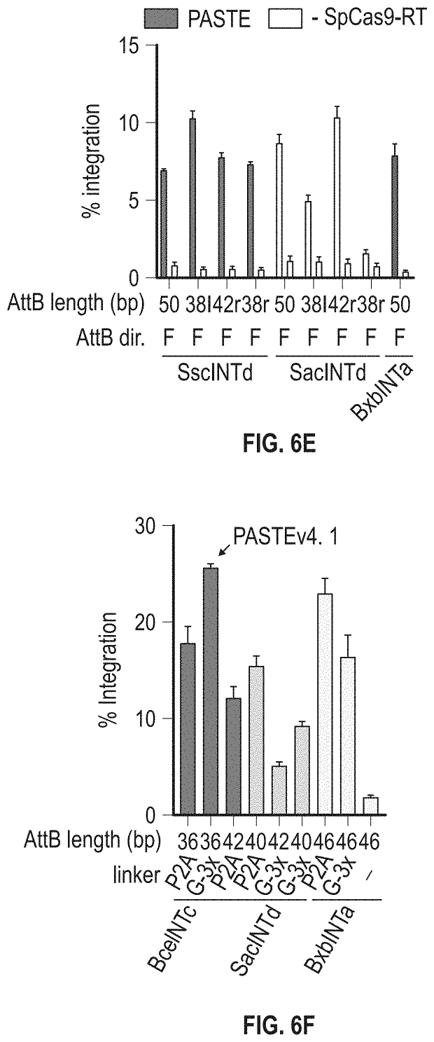

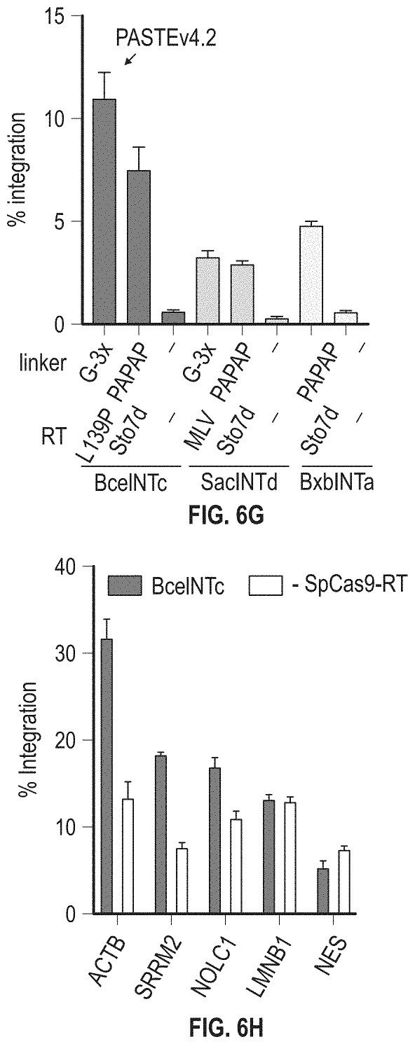

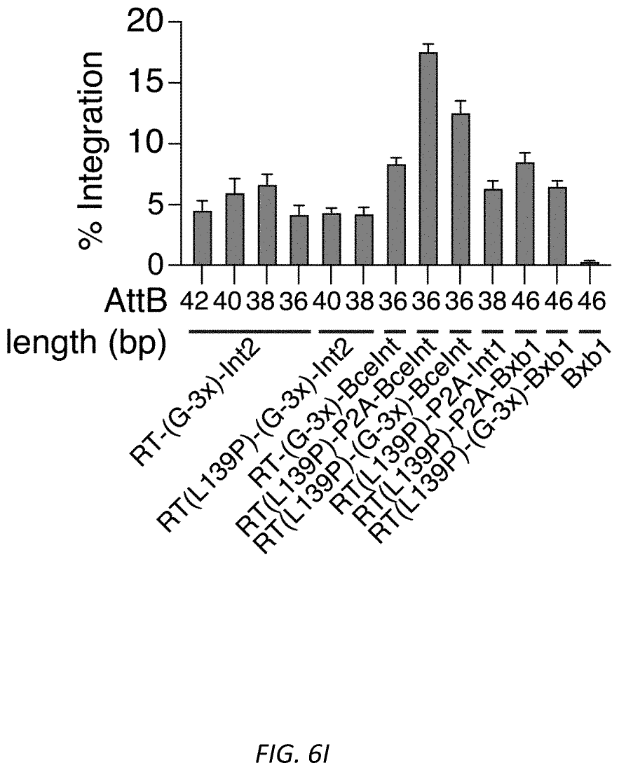

Integrase choice can have implications for integration activity. To identify novel integrases with improved activity in the PASTE system, bacterial and metagenomic sequences were mined for new phage associated serine integrases (FIG. 5A). Exploring over 10 TB worth of data from NCBI, JGI, and other sources, 27,399 novel integrases were found (FIG. 5B, FIG. 5C) and their associated attachment sites were annotated using a novel repeat finding algorithm that could predict potential 50 bp attachment sites with high confidence near phage boundaries. Table 8 above recites top scoring integrase enzyme amino acid sequences and the attB/attP nucleic acid sequences recognized by said integrase enzymes. Accordingly, for each row of Table 8, an integrase amino acid sequence is recited, followed by the corresponding attB and attP nucleic acid sequence to which said integrase binds. Analysis of the integrases sequences revealed that they fell into four distinct clusters: INTa, INTb, INTc, and INTd. About half of integrases (14,771) derive from metagenomic sequences, presumably from pro-phages, and 13,693 of the integrases specifically derive from human microbiome metagenomic samples. An initial screen of integrase activity using a reporter system revealed that a number of the integrases were highly active in HEK293FT cells with more activity than BxbINT, a member of the INTa family (FIG. 6A). Using the predicted 50 bp sequences encoded in attachment site-containing guide RNAs (atgRNAs) along with minicircles containing the complementary AttP sites, it was found that these integrases were compatible with PASTE but with lower efficiency than BxbINTa-based PASTE (FIG. 6B). It was hypothesized that this was because of their longer 50 bp AttB sequences and so truncations of these AttBs were explored in the hopes of finding more minimal attachment sites. Truncation screening on integrase reporters revealed that AttB truncations of all the integrases, including as short as 34 bp, were still active and many had more activity than BxbINTa (FIG. 6C). Upon porting these new shorter AttBs to atgRNAs for PASTE, it was found that a number of integrases had more activity in the PASTE system than BxbINT-based PASTE at the ACTB locus, including the integrase from B. cereues (BceINTc), N191352_143_72 stool sample from China (SscINTd), and N684346_90_69 stool sample from adult in China (SacINTd), while others like the integrase from B. cytotoxicus (BcytINTd) and S. lugdunensis (SluINTd) did not (FIG. 6A and FIG. 6D-FIG. 6E). Because of its superior efficiency when used with PASTE, BceINTc when used as PASTE is referred to as PASTEv4.1. Moreover, upon optimization of these integrases with different linkers and RT domains, it was found that BceINTc fused to SpCas9-RTSto7d or SpCas9-MLV-RT^{L139P} variant had the most activity, even higher than BxbINTa-based PASTE (FIG. 6G-FIG. 6I). The construct SpCas9-MLV-RT^{L139P}-BceINTc construct is referred to as PASTEv4.1. We then evaluated this optimized PASTEv4.1 and found that across a number of endogenous gene loci that it performed better than BxbINTa-based PASTE (FIG. 6H and FIG. 6J).

### Example 4

### Screening of Additional Integrases

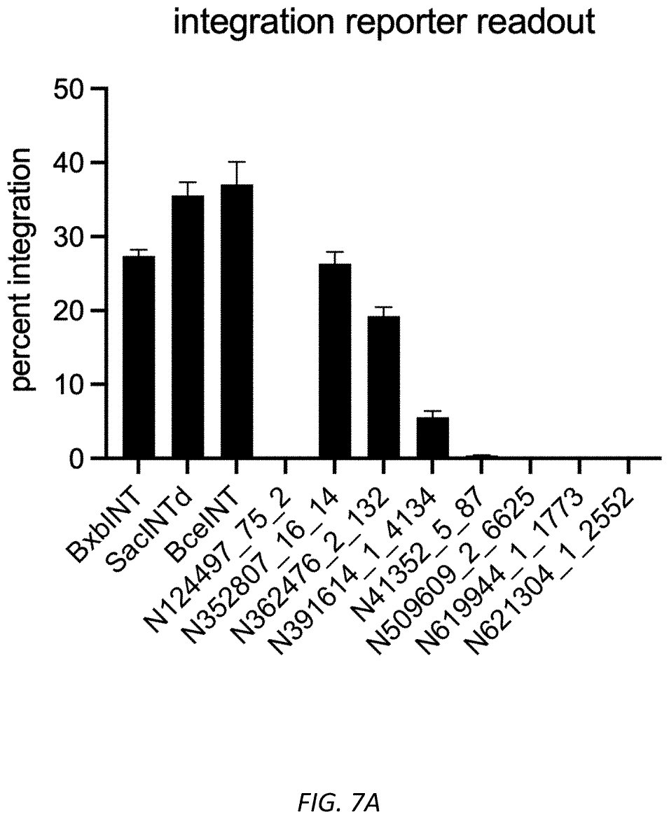

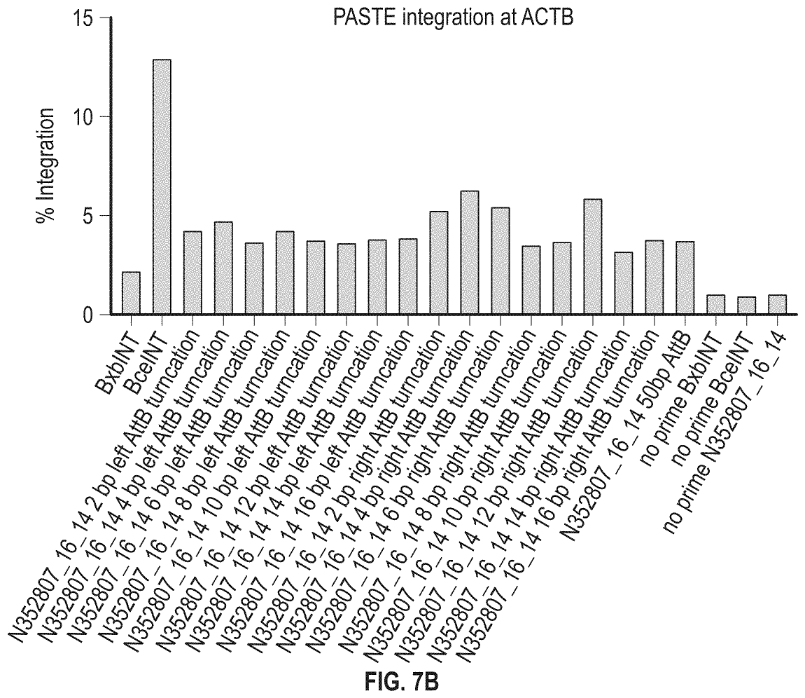

Ten additional integrases were tested for activity that were identified from the original discovery platform described above. The integrase amino acid sequences, nucleic acid sequences, and their corresponding attB and attP sites are recited in Table 9 below. As shown in FIG. 7A, several of the integrase displayed integrase activity in the HEK293 reporter assay described above. Integrases N352807_16_14, N362476_2_132, and N391614_1_4134 displayed measurable activity. The integrase N352807_16_14 was next tested in the PASTE system with integration at the ACTB gene locus, along with truncations of the attB site. The truncations tested were 2, 4, 6, 8, 10, 12, 14, or 16 base pair truncations from either the left or right of the attB sequence. As shown in FIG. 7B, the integrase N352807_16_14 achieved higher integration levels at the ACTB gene locus with all attB truncations compared to the BxbINT PASTE system.

TABLE 9
Integrase Amino Acid Sequences with Corresponding
attB/attP Nucleic Acid Sequences

| Integrase | Protein sequence | AttB | AttP |
|---|---|---|---|
| N124497_75_2 | MRAAIYARYSTEMQREASIEDQFRICRRLIEQNGW | GCATCTGCTGAT | CGCGGGTCTCA |
| | TTAQTYADAGLSGASHLRPGYQQMLQDARDGRF | AGCCTGGCCGTA | GCGGGGCATCG |
| | DVAVAEGLDRISRDQEHIAAFFKQLRFQGISIVTVA | GGGTCGGCGGC | CACCGAATTGCC |
| | EGEISELHIGLKGTMSSLFLKDLALKTHRGLEGRVQ | CTCATCAGGCTC | CCTGCTCAGAAC |
| | NGKSAGGVTYGYDVLRSIGSDGNVSTGDRTINEEQ | ATA (SEQ ID NO: | CGCA (SEQ ID |
| | ADIVRRIFQEYVDGHSPRAIAGRLNDEGIDGPRGR | 3176) | NO: 3186) |
| | GWGMSTIYGNWRRGTGILNNELYVGRLVWNRQ | | |
| | RFLKDPATGRRQARMNPPEQWVKKDVPDLRIVP | | |
| | NKLWEKVKARQKATRSTVISEGVVRSERAKRPAYL | | |
| | FSGLVKCGCCGGGFTLVGKTYYGCANARNKGTCD | | |
| | NKMTVRRDRLEDTVLGGLKDQLLHPDLIAAFVTEY | | |
| | QAEYNRLAGEITKDKSKAERKLAGVKRRIDQLVDRI | | |
| | CDGMFHESMKDKLTALEQEKAALETELATFDADV | | |
| | PVRLHPGLSDVYRAKVAKLTESLNNPDLRTAASEHI | | |
| | RSLISEIRMVPEGDSLQIELVGELAGLMALSQKSKA | | |
| | RGDATGCSITMVAGVGFEPTTFRL | | |
| | (SEQ ID NO: 3166) | | |
| N321537_3_247 | MSQKISAEHLCRGAVVYVRQSTMSQVVEHTESQN | ATCTCCTGGGTC | TTCTGCTGGAAG |
| | RQYALAESARAMGFASVTTIDDDLGRSGSGSVERP | GTTTGTGTTCAC | CGCTGGGCAGA |
| | GFQKLVASVCAGSVGAVFCLEASRLARNGRDWHH | CGGTGAACACA | CCGGTCTGCCCA |
| | LIDLCALVGTVVVDHDGIYDPRLVNDRLLLGLKGT | AACGACCCAGG | GCGCTTCCAGCA |
| | MSEYELSLLRQRGIAARDSKARRGELRFALPPGYC | AGAT (SEQ ID | GAA (SEQ ID |
| | WNELGQIEMDADERVAEAIRVLFRKFRELGSARQ | NO: 3177) | NO: 3187) |
| | VLLWAIQSDVKLPVTRHGPAGIRIEWSRPAYHTVV | | |
| | QILEHPVYAGAYVFGRTTHRTAVIDGRARKTEGHS | | |
| | KPMRDWSVLLRDHHPGYITWDQFEEHQRMIAEN | | |
| | THMKKRASRRSARGGRALLTGLARCGRCGRMMR | | |
| | VNYGTRAGHAHRYYCRGDEAHVGAFRCIGIGGIRI | | |
| | DKAVAAAILEAVSEHAVEAAVRAAQQTSRAGDDV | | |
| | RRALACELEEARYDASLAARRYEVVDPTKRLVAREL | | |
| | EGRWNTALERVAHLEERAALLDREVASRPAIDQTQ | | |
| | LMTLARDLPAAWNAPGTDMRTKQRLTRILIQEVVI | | |
| | DLDDITNEAVATVHWTGGRHTELRVARVNVGRYP | | |
| | SSQHFSAVEVMRKLGGQWPDRELAVTMNRMRC | | |
| | KSPDSRTWTTVRVTEMRERLGIAPFDPTASHAETIS | | |
| | VEEASRRLGIYGSSIHRLIREGILPATQLMPSAPWQ | | |
| | VPVAALDSDAVRAGVQAIKDRRPPHFKARQEADK | | |
| | SLKLPGF (SEQ ID NO: 3167) | | |
| N337281_45_252 | MTAVIYARYSSDSQREASIEGQLRDCKDYAEKNGIT | GCGTAGATCAC | AAGTCGAATAC |
| | VVGTYIDRAYSAKTDDRPDFQRMIKDSGKKIFDVV | GGCGGTCATGG | GCGCCAAGGAG |
| | LVWKLDRFARNRFDAVNYKYQLEKNGVHLVSVME | TTGCAACCATGA | GTGCACCTCCTT |
| | PISQGPEGIMVESMLIGMAEYYSAELALKVARGER | CCGCCGTGATCT | GGCGCGTATTC |
| | ENALQCKYNGGVVPLGFTIGKEDRLYHIDPETAPIV | ACGC (SEQ ID | GACTT (SEQ ID |
| | QEIFTRYADGEPAEKIAASLNERGLRTRTGKPFVKN | NO: 3178) | NO: 3188) |
| | SFFQIFRNRRYIGEYRYKDIVTPGGIPAIVDQDLFDR | | |
| | VQQRFEQNRIAHGRPAKEDVRYLLTTKLFCGKCGT | | |
| | LMGGESGTSHMGNTYYYYKCGNAKRHGKAHCDL | | |
| | KAIRKEPLERFVVETAIKVIFSDEIIEQLIDLIMEAQQ | | |
| | QENTRLPVLKDQLRDTEKRLANLLEAIEQGILTPTTK | | |
| | QRLDELEARKEALNTSILEEELKKPVLTREWMRFW | | |
| | FEKFRKGDMRDMEHQRQIIDTFVNSVYVFDDRVV | | |
| | LNFNFTDDSKTISREEVLGSSAVDNAPPHKNPQTF | | |
| | VWGFLFCVGGEAQDENRAREQSDTIVKGRIPPSW | | |
| | (SEQ ID NO: 3168) | | |
| N352807_16_14 | MKVIGYARLSRATEESTSIARQRQAIQDTARQRGW | GCTTACACTTTG | TCCCCGGACATG |
| | ELVRIETDNDVSATRTRLDRPGLNAVRDAIAAGDA | GTCACACTCGGC | CTCGTGTTCGCG |
| | DAVLVWKLDRIARSVVDFGLLLDDGLQIVSCMEPL | ACCGCGGATGA | GTGCTGCAGCC |
| | DTTTPMGRAMAEILQVFAAMEARTIGQRVSSSRK | GACTAAAGTGTA | GATCATGTTCGT |
| | HLATIGRFPGGQPPYGYRSVPMPGGTGRTLEVDP | CGT (SEQ ID NO: | GTT (SEQ ID |
| | DEAAIVRKAVDAVLGGESMYAVLKELDEAGVRTRR | 3179) | NO: 3189) |
| | GLSWTLTTFPRVLCSEAMLGRARFQGKTVRDDNG | | |
| | LPLTPFPPILTLEEHDQLVARFAPDAEHAERTRAGR | | |
| | RKASRLLSGLLYCESCNSRLVVRTNNHPSRPPVYFC | | |
| | PSRGTGVVCTARVSGLASKLEELVTDTFLDLYGSAP | | |
| | FTVEHTSVQERSDVALVEAAIRDTTDELREPDADV | | |
| | MALVDRLNSLRAERERLDKLPTAPVVERVPTGETV | | |
| | AQVWHRSDYRAQRDLLIRAGFYGVLRPAKRRGYW | | |
| | DDDRFDIQWDVDDVLSLDPNDVEAQSRGPITV | | |
| | (SEQ ID NO: 3169) | | |
| N362476_2_132 | MTSPLPAAIYLRVSGKHQAEGRFGLAAQEHECRAY | GTTTATACGCGT | TACTCATTCATC |
| | AARVGLRVARVYVDVISGARDERAQFTQLLADAA | GCACACCTAATC | TGGCAGCCGTA |
| | GYSVVILGVQDRLARDVPLSYAMLGALQRAGLRV | ACCTATAGGTGT | GGTGATTAGGT |
| | HSALEGPLDLEDDGHALNFGIRAVIADQERRRITAR | TCAGTCGTATAA | GTGCCTTCATCA |
| | MYGGKLAKVRDRGQPVAPIRAYGWKDGAPDPET | AG (SEQ ID NO: | TGTT (SEQ ID |
| | GARVAWIFEQVERRGLNQVLDELERLGVPSPTGRP | 3180) | NO: 3190) |
| | RWGKTALLNLIRNPLYRGEYGYGRKGERLTLAVPAL | | |
| | VTPEQWARTNAAVARRFKGSGRAGTLAHIYHLQG | | |
| | VARCGECGSTMSAHSPTPRDGAVRHRYYHCRGTL | | |
| | KIAGRRCEHRRSYPIDVLHQVVAEGLAVLLSEPKLLA | | |
| | EAVARGVHERPGSGPGRVATELARLDAEWERWK | | |
| | GALRAGAITPEELAAERRRIDAARAALSTVEAPTPL | | |
| | DVETWVARTTRAVADLPLGEALRVAGIGVRVHAG | | |
| | GAVEFVVTP (SEQ ID NO: 3170) | | |
| N391614_1_4134 | MTTRKRAVIYTRISKDRNGESLAVDRQEKACRKLAK | CTAGGCGTCACC | GCCAGGTCGGC |
| | LAEMEVVDIFTDNDISAYNGKRRDGFEDMLVAIES | TGTACATGCTGT | GGCCACCTGCTC |
| | GRADVVLCQHTDRLYRRLADLVRLCQAGPNLLIKTI | GGCGAGCATGT | GCCGTACGCGG |
| | QGGDLDLSTSTGKMLAQILGSVQEQESAHHSERR | ATAGGGGACGC | TGGCCGCCCGG |
| | KSAYVQRAELGVFHNQGNRSFGYTRDGQPIEPEAT | CTAA (SEQ ID | GTGCC (SEQ ID |
| | MYRDAVADLLEGTSLRAIARQWNASGVTTTVAGA | NO: 3181) | NO: 3191) |
| | TRKLKGKEYVVKGVWSSTRIRRLLLNPRYAGIKTHL | | |
| | GKEVETQANWTPLIDEETYRRVVAELSDPTRLKVTS | | |
| | FEKKHVGSGVYVCGVCGAPMQISFPGPGRSQGRK | | |
| | YVCSAHSCVIRGGDPVDDYVENLVVERLSRPDAGL | | |
| | LIAERGVDVGKLQDQRAGWVTKLDRLVDLLDDGT | | |
| | LDGPKARRRAAEYKAEINKIDSQLAQAARTSPTAAL | | |
| | LAAGKELRMRWPKLTAGVRGEIISEIATVTINRCGK | | |
| | GKRFDPHAVVDGKVVLDVQWKVDSP (SEQ ID | | |
| | NO: 3171) | | |
| N41352_5_87 | MRVLAVKRISRDTEQSSALARQEVQLSKAIREGHH | CGTTTGGAATCC | CCCCGGGTCAG |
| | TVAGWVEDATVSGAVNLDKRPSLGQWLTAPLIHE | CTAACATCCGAC | GGTGACGCCCG |
| | WDGMMVTEQDRITRDDLHWAAFVGWVLENGK | ACGTCGGACTTG | AGGTGTCGGAT |
| | TIVVLDDPSFDITTPNGRLIASVKAAQAANYRNSVR | CGATAGCCCAAA | GTTAGGGATTCC |
| | TKKLNQLEWYREERLWSGGSWPFGYRAIRVLHRE | CC (SEQ ID NO: | AAACG (SEQ ID |
| | ALRWRLGIDPVTGPLIREAYDRLVNKGHSIRMIVLD | 3182) | NO: 3192) |
| | WNARGILTARDYQRHVKALEGGEQAEAAVKGTQ | | |
| | WSNTVLRVVLTNPALMGYAVYKGEIQKKAGLPVQ | | |
| | WADPILTLEEFEKLQEVLARRGGANRNITPRTTDW | | |
| | AGIFYCACGSVYYSNSAPRKLKDGSVRRHDYYLCKT | | |
| | RQVGTRCAYVTSWPLEILRTHLEDAFLSNAGDLEI | | |
| | MTRTYIPGSDRSADIRQLQEALENLTGNLIHLKPGS | | |
| | AGATAVLKAMEEHEAALAELEALPVIPSRWQESGT | | |
| | GETFRQFWERNPSWEVRCDFLRKTGVRLYVAGNP | | |
| | KAPDLDLFLPDDLQQRIINAVSDTVEPGALEAAGR | | |
| | QADDAMAERRASDAALREAAMEATRGTRDLTR | | |
| | (SEQ ID NO: 3172) | | |
| N509609_2_6625 | MTRAALYARFSSDQQKDRSIADQISLCRDLCAREG | ACCCGAGACATA | GGAAAGTGTAA |
| | MTVISTFEDRAISGSAAVNRPGFQALMQAAESRLF | GGTGACAGTTC | CCCATGTGTCCG |
| | DVIVAEDMDRIFRDQADYHNVRKRLDFLGITLHTA | GTACGAACTGTC | GTACCGGACAC |
| | TGKIGTIDGALRALMSEQFIENLRVHTRRGLEGVLR | ACCTATGTCTCG | ATGGGTTACACT |
| | DGRHAGGRAYGYRAIKDKPGELEIVEDEAEIVRQIF | GGT (SEQ ID | TTCC (SEQ ID |
| | SDYVAGKTPRQIAHDLNNRSVRPPRGRLWNGSTI | NO: 3183) | NO: 3193) |
| | NGNVARGGGMILNDLYAGRIVWNKVRMVKDPA | | |
| | TGKRLSRPNPKEQYRIVEAPHLRIIDDATFKAAQGIK | | |
| | AERRRDATPASAQRARAPKRVFSGLIRCGSCGGG | | |
| | MSSIGSDRKGLRLQCSAHRESGSCDNGRRIYLSDIE | | |
| | ALAIKGLRQHLAHPEVIAEFVDAYNAERKRLKKEAS | | |
| | TERARLERRLGEIEREMRRIVDSIVVTGMPPEQFVA | | |
| | RMQELEAEKTKVMAGLERAKETDNVIALHPKALD | | |
| | RYKDAVVELADELKRGTPTEFATVRELVTAIIVHASP | | |
| | SRPGGAGTRANAEDDRNVRIDIKGRLAALCGNPAL | | |
| | FPNMAMSG (SEQ ID NO: 3173) | | |
| N619944_1_1773 | MQVRGVTPFVSAVGAEKLDTVRVGVYARQSKARP | CGAACGCGAGC | ACTGCCCATGAA |
| | DSSEASPEAQLAAGQMLAASRGWEVVHTFKDVG | AGGAACCAGGA | GCTCAGCCGTCG |
| | RSGWDPNAVRPEFEALMEAVRAGEVDVVIVNELS | AACGTCTCCTGG | CGCGACGGCTG |
| | RLTRKGAHDALEIDKEFKEYGVRFVSVLEPFLDTSN | TTCCTGCTCGCG | AGCTTCATGGTC |
| | PIGVAIFALIAALAKQDSDIKAERLRGAKDEIAAVGG | TTCG (SEQ ID | CCA (SEQ ID |
| | RHSSSPPFGMRAVREKIGSLVVSVLEPDEDNPDHV | NO: 3184) | NO: 3194) |
| | ALVKRMAAMSADGISDNKIATTFTEEEIPAPGEAE | | |
| | RRATPKRMESIKRRRVSDKERPIQWRAQTVKWILS | | |
| | HPAIGGFASERVKRGKAYENVIARNLVGKPLAPHR | | |
| | GIIPGAQWLDLQEKRKAGTQPGRKSGGNAVPTLLS | | |
| | GWRFATCGVCDGALGQTPGNGDGSYMCANPKG | | |
| | HGGLGIKRSELDDYVARRVWARLENSDMDDPEDR | | |
| | VWVEAAAQRFALQKDLSSAQEERRETELHLDHVR | | |
| | QSIAELQEDRKRGLYRGRDELNTWRATLEQYRAYE | | |
| | DQCVARLTELEEEASATIRIPVEWTEPGEDPIGKGS | | |
| | PWASWDIFERRAFLDLFLSGVSVGRGRDPETRKYI | | |
| | AVEDRVTLHWRPLPADDETDED | | |
| | (SEQ ID NO: 3174) | | |
| N621304_1_2552 | MKAVIYARVSTQDQALGFSLATQKELCEKKARALG | TTTCCGGTGGTA | AAAGGTGCCCC |
| | ALEVEVVEDAYTGTELNRPSLDYVRQLVATGKVDL | CCAGCCTGGGA | CGTCAATAAACA |
| | VVVYDPDRLSRNLTDLLILCREFDKAGVRLEFVNFD | GGAATCCTGCTC | CTCATGTTTATC |
| | WQKTPQGMLFLQMRGAFAEFEHALIKERTQRGK | TGGGGAAGAAG | TCCTTCAACATC |
| | DKKAASGKIRCYAKPFGYDWDAEGDTLVINPQEAE | AAAG (SEQ ID | TCT (SEQ ID |
| | IVKQMFEWLTDPLEPLTPWQITQKLGQVYPQGPR | NO: 3185) | NO: 3195) |
| | GKGWVHSSVVRMLKNPVYTGRLRRKDEQPDWKP | | |
| | VLVPAIIDQVTFMRAQEMLARSKHFNPKTTRRRFL | | |
| | LQRLLVCGECGRRLTVVTHNNPQKAKYSYYTCPGR | | |
| | YPTKFDDRGRVGRCGLPPWRTEEIDKTVWDTIASII | | |
| | KNPELFYQYITSEKLETASIPRRRLEEARKRLEQVQR | | |
| | VIERIDRAYFILEALPEEDYKRYRAEQEGELIRIEEDIK | | |
| | RLEAVINAQEQVQKGVEFLRQYAENLARSVDELNF | | |
| | FQKQNITRELVQRVKIYADGSLEIEGYFNLPFTGPG | | |
| | KNVPDHCEELTTLTTQKNLKEPLSSSVLTCTHRKKG | | |
| | HPKVTEGIIHLTILAV (SEQ ID NO: 3175) | | |

One skilled in the art will appreciate further features and advantages of the disclosure based on the above-described embodiments. Accordingly, the disclosure is not to be limited by what has been particularly shown and described, except as indicated by the appended claims.

LENGTHY TABLES
The patent application contains a lengthy table section. A copy of the table is available in electronic form from the USPTO web site (https://seqdata.uspto.gov/?pageRequest=docDetail&DocID=US20230272435A1). An electronic copy of the table will also be available from the USPTO upon request and payment of the fee set forth in 37 CFR 1.19(b)(3).

1. An integrase or fragment thereof comprising an amino acid sequence that is at least 80% identical to an amino acid sequence set forth in any one of SEQ ID NOs: 1-16.

2. The integrase of claim 1, wherein the integrase fragment comprises integrase, recombinase, or transposase activity.

3. (canceled)

4. The integrase of claim 1, wherein the integrase binds to nucleic acid attachment sites attB and attP, other recognition site pairs, or any pseudosites in a human genome.

5. (canceled)

6. (canceled)

7. The integrase of claim 6, wherein the attB and/or attP nucleic acid sequence is truncated by 1 to 32 nucleotides from one or both of the 5′ end and 3′ end.

8. The integrase of claim 4, wherein the integrase binds to any one of the attB nucleic acid sequences selected from the group consisting of SEQ ID NOs: 17, 19, 21, 23, 25, 27, 29, 31, 33, 35, 37, 39, 41, 43, 45, and 47, or a nucleic acid sequence with at least 80% identity to any one of the attB nucleic acid sequences selected from the group consisting of SEQ ID NOs: 17, 19, 21, 23, 25, 27, 29, 31, 33, 35, 37, 39, 41, 43, 45, and 47.

9. The integrase of claim 4, wherein the integrase binds to any one of the attP nucleic acid sequences selected from the group consisting of SEQ ID NOs: 18, 20, 22, 24, 26, 28, 30, 32, 34, 36, 38, 40, 42, 44, 46, and 48, or a nucleic acid sequence with at least 80% identity to any one of the attP nucleic acid sequences selected from the group consisting of SEQ ID NOs: 18, 20, 22, 24, 26, 28, 30, 32, 34, 36, 38, 40, 42, 44, 46, and 48.

10. The integrase of claim 4, wherein:
a) the integrase or fragment thereof comprises an amino acid sequence that is at least 80% identical to an amino acid sequence set forth in SEQ ID NO: 1, wherein the integrase binds to the attB nucleic acid set forth in SEQ ID NO: 17 and the attP nucleic acid set forth in SEQ ID NO: 18;
b) the integrase or fragment thereof comprises an amino acid sequence that is at least 80% identical to an amino acid sequence set forth in SEQ ID NO: 2, wherein the integrase binds to the attB nucleic acid set forth in SEQ ID NO: 19 and the attP nucleic acid set forth in SEQ ID NO: 20;
c) the integrase or fragment thereof comprises an amino acid sequence that is at least 80% identical to an amino acid sequence set forth in SEQ ID NO: 3, wherein the integrase binds to the attB nucleic acid set forth in SEQ ID NO: 21 and the attP nucleic acid set forth in SEQ ID NO: 22;
d) the integrase or fragment thereof comprises an amino acid sequence that is at least 80% identical to an amino acid sequence set forth in SEQ ID NO: 4, wherein the integrase binds to the attB nucleic acid set forth in SEQ ID NO: 23 and the attP nucleic acid set forth in SEQ ID NO: 24;
e) the integrase or fragment thereof comprises an amino acid sequence that is at least 80% identical to an amino acid sequence set forth in SEQ ID NO: 5, wherein the integrase binds to the attB nucleic acid set forth in SEQ ID NO: 25 and the attP nucleic acid set forth in SEQ ID NO: 26;
f) the integrase or fragment thereof comprises an amino acid sequence that is at least 80% identical to an amino acid sequence set forth in SEQ ID NO: 6, wherein the integrase binds to the attB nucleic acid set forth in SEQ ID NO: 27 and the attP nucleic acid set forth in SEQ ID NO: 28;
g) the integrase or fragment thereof comprises an amino acid sequence that is at least 80% identical to an amino acid sequence set forth in SEQ ID NO: 7, wherein the integrase binds to the attB nucleic acid set forth in SEQ ID NO: 29 and the attP nucleic acid set forth in SEQ ID NO: 30;
h) the integrase or fragment thereof comprises an amino acid sequence that is at least 80% identical to an amino acid sequence set forth in SEQ ID NO: 8, wherein the integrase binds to the attB nucleic acid set forth in SEQ ID NO: 31 and the attP nucleic acid set forth in SEQ ID NO: 32;
i) the integrase or fragment thereof comprises an amino acid sequence that is at least 80% identical to an amino acid sequence set forth in SEQ ID NO: 9, wherein the integrase binds to the attB nucleic acid set forth in SEQ ID NO: 33 and the attP nucleic acid set forth in SEQ ID NO: 34;
j) the integrase or fragment thereof comprises an amino acid sequence that is at least 80% identical to an amino acid sequence set forth in SEQ ID NO: 10, wherein the integrase binds to the attB nucleic acid set forth in SEQ ID NO: 35 and the attP nucleic acid set forth in SEQ ID NO: 36;
k) the integrase or fragment thereof comprises an amino acid sequence that is at least 80% identical to an amino acid sequence set forth in SEQ ID NO: 11, wherein the integrase binds to the attB nucleic acid set forth in SEQ ID NO: 37 and the attP nucleic acid set forth in SEQ ID NO: 38;
l) the integrase or fragment thereof comprises an amino acid sequence that is at least 80% identical to an amino acid sequence set forth in SEQ ID NO: 12, wherein the integrase binds to the attB nucleic acid set forth in SEQ ID NO: 39 and the attP nucleic acid set forth in SEQ ID NO: 40;
m) the integrase or fragment thereof comprises an amino acid sequence that is at least 80% identical to an amino acid sequence set forth in SEQ ID NO: 13, wherein the integrase binds to the attB nucleic acid set forth in SEQ ID NO: 41 and the attP nucleic acid set forth in SEQ ID NO: 42;
n) the integrase or fragment thereof comprises an amino acid sequence that is at least 80% identical to an amino acid sequence set forth in SEQ ID NO: 14, wherein the integrase binds to the attB nucleic acid set forth in SEQ ID NO: 43 and the attP nucleic acid set forth in SEQ ID NO: 44;
o) the integrase or fragment thereof comprises an amino acid sequence that is at least 80% identical to an amino acid sequence set forth in SEQ ID NO: 15, wherein the integrase binds to the attB nucleic acid set forth in SEQ ID NO: 45 and the attP nucleic acid set forth in SEQ ID NO: 46; or
p) the integrase or fragment thereof comprises an amino acid sequence that is at least 80% identical to an amino acid sequence set forth in SEQ ID NO: 16, wherein the integrase binds to the attB nucleic acid set forth in SEQ ID NO: 47 and the attP nucleic acid set forth in SEQ ID NO: 48.

11. The integrase of claim 4, wherein any one of the attB nucleic acid sequences selected from the group consisting of SEQ ID NOs: 17, 19, 21, 23, 25, 27, 29, 31, 33, 35, 37, 39, 41, 43, 45, and 47 is truncated by 1 to 32 nucleotides from one or both of the 5′ end and 3′ end.

12. The integrase of claim 4, wherein any one of the attP nucleic acid sequences selected from the group consisting of SEQ ID NOs: 18, 20, 22, 24, 26, 28, 30, 32, 34, 36, 38, 40, 42, 44, 46, and 48 is truncated by 1 to 32 nucleotides from one or both of the 5′ end and 3′ end.

13. The integrase of claim 1, wherein the integrase is linked to a DNA binding domain via a linker.

14. The integrase of claim 13, wherein the DNA binding domain is a DNA binding nuclease.

15. The integrase of claim 14, wherein the DNA binding nuclease is selected from the group consisting of a zinc finger nuclease (ZFN), a transcription-activator like effector nuclease (TALEN), an argonaute, and an RNA-guided nuclease.

16. The integrase of claim 15, wherein the RNA-guided nuclease comprises a CRISPR nuclease.

17. The integrase of claim 16, wherein the CRISPR nuclease is Cas9 or Cas12.

18. The integrase of claim 16, wherein the CRISPR nuclease comprises nickase activity.

19. The integrase of claim 16, wherein the CRISPR nuclease is selected from Cas9-D10A, Cas9-H840A, and Cas12a/b nickase.

20. The integrase of claim 14, wherein the DNA binding nuclease and/or the integrase are linked to a reverse transcriptase domain via a linker.

21. The integrase of claim 20, wherein the reverse transcriptase domain is selected from the group consisting of Moloney Murine Leukemia Virus (M-MLV) reverse transcriptase domain, transcription xenopolymerase (RTX), avian myeloblastosis virus reverse transcriptase (AMV-RT), and Eubacterium rectale maturase RT (MarathonRT).

22. (canceled)

23. The integrase of claim 21, wherein the M-MLV reverse transcriptase domain comprises one or more mutations selected from the group consisting of D200N, T306K, W313F, T330P, L603W, and L139P.

24. The integrase of claim 13, wherein the linker is cleavable or non-cleavable.

25. (canceled)

26. The integrase of claim 13, wherein the linker can be replaced by two associating binding domains of the DNA binding nuclease linked to a reverse transcriptase.

27. (canceled)

28. (canceled)

29. (canceled)

30. (canceled)

31. A polynucleotide comprising a nucleic acid sequence encoding the integrase of claim 1.

32. A vector comprising the nucleic acid sequence of claim 31.

33. A host cell comprising the vector of claim 32.

34. A fusion protein comprising DNA binding nuclease, a reverse transcriptase domain, and an integrase or fragment thereof, wherein the DNA binding nuclease is linked to the reverse transcriptase domain and/or the integrase or fragment thereof via a linker,
wherein the integrase or fragment thereof comprises an amino acid sequence that is at least 80% identical to an amino acid sequence set forth in any one of SEQ ID NOs: 1-16.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. A method of site-specific integration of a nucleic acid into a cell genome, the method comprising:
(a) incorporating an integration site at a desired location in the cell genome by introducing into the cell:
i. a DNA binding nuclease linked to a reverse transcriptase domain, wherein the DNA binding nuclease comprises a nickase activity; and
ii. a guide RNA (gRNA) comprising a primer binding sequence linked to an integration sequence, wherein the gRNA interacts with the DNA binding nuclease and targets the desired location in the cell genome, wherein the DNA binding nuclease nicks a strand of the cell genome and the reverse transcriptase domain incorporates the integration sequence of the gRNA into the nicked site, thereby providing the integration site at the desired location of the cell genome; and
(b) integrating the nucleic acid into the cell genome by introducing into the cell:
i. a DNA or RNA strand comprising the nucleic acid linked to a sequence that is complementary or associated to the integration site; and
ii. an integrase or fragment thereof comprising an amino acid sequence that is at least 80% identical to an amino acid sequence set forth in any one of SEQ ID NOs: 1-16, wherein the integrase or fragment thereof incorporates the nucleic acid into the cell genome at the integration site by integration, recombination, or reverse transcription of the sequence that is complementary or associated to the integration site, thereby introducing the nucleic acid into the desired location of the cell genome of the cell.

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

103. (canceled)

104. (canceled)

105. (canceled)

106. (canceled)

107. An integrase or fragment thereof comprising an amino acid sequence that is at least 80% identical to any one of the integrase amino acid sequences set forth in Table 8.

108. (canceled)

109. (canceled)

110. (canceled)

111. (canceled)

112. (canceled)

113. (canceled)

114. (canceled)

115. (canceled)

116. (canceled)

117. (canceled)

118. A fusion protein comprising DNA binding nuclease, a reverse transcriptase domain, and an integrase or fragment thereof, wherein the DNA binding nuclease is linked to the reverse transcriptase domain and/or the integrase or fragment thereof via a linker,
wherein the integrase or fragment thereof comprises an amino acid sequence that is at least 80% identical to any one of the integrase amino acid sequences set forth in Table 8.
